SEQUENCE LISTING (19 sequences)

SEQ ID NO: 1 - protein, 10 aa, Unknown
  GnRH sequence
  misc_feature (1)..(1): Xaa is pyroGlu
  misc_feature (10)..(10): Xaa is Gly-NH2
XHWSYGLRPX

SEQ ID NO: 2 - protein, 10 aa, Unknown
  GnRH sequence
EHWSYGLRPG

SEQ ID NO: 3 - DNA, 12 bp, Artificial Sequence
  Synthetic oligonucleotide
tagtagtctaga

SEQ ID NO: 4 - DNA, 2283 bp, Artificial Sequence
  GnRH8-Hinge-Thelp-LFn-Thelp-hinge-GnRH8
aagcttaccatggatgcaatgaagagagggctctgctgtgtgctgctgctgtgtggagcagtcttcgtttcgcccagcggtaccggatccgaacactggtcttacgggctgaggcctggcagtggatccgagcattggtcttacggcctgagacctggcggcagttctgaacactggtcatacggactcagaccaggaagcggctctgagcattggtcttatggcctgcgccctggcggcagttctgaacactggtcatacggactcagaccaggaagcggctctgagcattggtcttatggcctgcgccctggcggcagttctgaacattggtcttacggactgaggcctggaagtggatccgagcactggtcttatggcctccgccctggcggcagttctacttgtcctccctgcccagctccgagcggctctactgctgcccaaattaccgcaggtatcgcgcttcatcagtctaatctcaacggcagttctaaacttattcctaatgcttctctcatcgaaaactgtactaaggcggagttgagcggctctgcggagttgggggagtacgaaaagctactcaactcggtgcttgaacccatcggcagttctgaatatctcaacaaaatccaaaatagcctaagtacagagtggagtccttgtagcgtaacgagcggctctgtcagtcccttctatagctacatcaaaacaagtaatattctggcagatgacgtaggcagttctgctaaagccgtagcagcgtggacactaaaggctgccgcaagcggctccgccggcggccacggcgatgtgggcatgcacgtgaaagagaaagagaaaaacaaagatgagaacaagcggaaagatgaagagcggaacaaaacccaggaagagcacctgaaggaaatcatgaaacacatcgtgaaaatcgaagtgaaaggcgaggaagctgtgaaaaaggaggccgctgaaaagctgctcgagaaagtgcccagcgatgtgctggagatgtacaaagccatcggcggcaaaatctacatcgtggatggcgatatcaccaaacacatcagcctggaagccctgagcgaagataagaaaaagatcaaagacatctacggcaaagatgctctgctccacgaacactacgtgtacgccaaagaaggctacgaacccgtgctcgtgatccagagcagcgaagattacgtggaaaacaccgaaaaggccctgaacgtgtactacgaaatcggcaagatcctgagccgggatatcctgagcaaaatcaaccagccctaccagaaattcctggatgtgctgaacaccatcaaaaacgccagcgatagcgatggccaggatctgctcttcaccaaccagctgaaggaacaccccaccgacttcagcgtggaattcctggaacagaacagcaacgaggtgcaggaagtgttcgccaaagctttcgcctactacatcgagccccagcaccgggatgtgctgcagctgtacgcccccgaagctttcaactacatggataaattcaacgaacaggaaatcaacctggccggcatgactgctgcccaaattaccgcaggtatcgcgcttcatcagtctaatctcaacggcagttctaaacttattcctaatgcttctctcatcgaaaactgtactaaggcggagttgagcggctctgcggagttgggggagtacgaaaagctactcaactcggtgcttgaacccatcggcagttctgaatatctcaacaaaatccaaaatagcctaagtacagagtggagtccttgtagcgtaacgagcggctctgtcagtcccttctatagctacatcaaaacaagtaatattctggcagatgacgtaggcagttctgctaaagccgtagcagcgtggacactaaaggctgccgcaagcggctccacttgtcctccctgcccagctccgagcggctctgaacactggtcttacgggctgaggcctggcagtggatccgagcattggtcttacggcctgagacctggcggcagttctgaacactggtcatacggactcagaccaggaagcggctctgagcattggtcttatggcctgcgccctggcggcagttctgaacactggtcatacggactcagaccaggaagcggctctgagcattggtcttatggcctgcgccctggcggcagttctgaacattggtcttacggactgaggcctggaagtggatccgagcactggtcttatggcctccgccctggcggcagttcttagtagtctaga

SEQ ID NO: 5 - protein, 27 aa, Artificial Sequence
  Start-TPA
MDAMKRGLCCVLLLCGAVFVSPSGTGS

SEQ ID NO: 6 - protein, 101 aa, Artificial Sequence
  8 copies GnRH+spacers
EHWSYGLRPGSGSEHWSYGLRPGGSSEHWSYGLRPGSGSEHWSYGLRPGGSSEHWSYGLRPGSGSEHWSYGLRPGGSSEHWSYGLRPGSGSEHWSYGLRPG

SEQ ID NO: 7 - protein, 8 aa, Artificial Sequence
  Hinge
TCPPCPAP

SEQ ID NO: 8 - protein, 17 aa, Artificial Sequence
  CDV T-help
KLIPNASLIENCTKAEL

SEQ ID NO: 9 - protein, 16 aa, Artificial Sequence
  Influenza Hemaglutinin T-help
GALNNRFQIKGVELKS

SEQ ID NO: 10 - protein, 223 aa, Artificial Sequence
  Start LFn - End LFn
QEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINL

SEQ ID NO: 11 - DNA, 2739 bp, Artificial Sequence
  GnRH8-Hinge-Thelp-LFn-HBc-Thelp-hinge-GnRH8
aagcttaccatggatgcaatgaagagagggctctgctgtgtgctgctgctgtgtggagcagtcttcgtttcgcccagcggtaccggatccgaacactggtcttacgggctgaggcctggcagtggatccgagcattggtcttacggcctgagacctggcggcagttctgaacactggtcatacggactcagaccaggaagcggctctgagcattggtcttatggcctgcgccctggcggcagttctgaacactggtcatacggactcagaccaggaagcggctctgagcattggtcttatggcctgcgccctggcggcagttctgaacattggtcttacggactgaggcctggaagtggatccgagcactggtcttatggcctccgccctggcggcagttctacttgtcctccctgcccagctccgagcggctctactgctgcccaaattaccgcaggtatcgcgcttcatcagtctaatctcaacggcagttctaaacttattcctaatgcttctctcatcgaaaactgtactaaggcggagttgagcggctctgcggagttgggggagtacgaaaagctactcaactcggtgcttgaacccatcggcagttctgaatatctcaacaaaatccaaaatagcctaagtacagagtggagtccttgtagcgtaacgagcggctctgtcagtcccttctatagctacatcaaaacaagtaatattctggcagatgacgtaggcagttctgctaaagccgtagcagcgtggacactaaaggctgccgcaagcggctccgccggcggccacggcgatgtgggcatgcacgtgaaagagaaagagaaaaacaaagatgagaacaagcggaaagatgaagagcggaacaaaacccaggaagagcacctgaaggaaatcatgaaacacatcgtgaaaatcgaagtgaaaggcgaggaagctgtgaaaaaggaggccgctgaaaagctgctcgagaaagtgcccagcgatgtgctggagatgtacaaagccatcggcggcaaaatctacatcgtggatggcgatatcaccaaacacatcagcctggaagccctgagcgaagataagaaaaagatcaaagacatctacggcaaagatgctctgctccacgaacactacgtgtacgccaaagaaggctacgaacccgtgctcgtgatccagagcagcgaagattacgtggaaaacaccgaaaaggccctgaacgtgtactacgaaatcggcaagatcctgagccgggatatcctgagcaaaatcaaccagccctaccagaaattcctggatgtgctgaacaccatcaaaaacgccagcgatagcgatggccaggatctgctcttcaccaaccagctgaaggaacaccccaccgacttcagcgtggaattcctggaacagaacagcaacgaggtgcaggaagtgttcgccaaagctttcgcctactacatcgagccccagcaccgggatgtgctgcagctgtacgcccccgaagctttcaactacatggataaattcaacgaacaggaaatcaacctggccggcatgatcgacattgacacgtataaagaatttggagcttctgtggagttactctcttttttgccttctgacttctttccttctattcgtgatctcctcgacaccgcctctgctctgtatcgggaggccttagagtctccggaacattgttcacctcaccatacagcactaaggcaagctattctgtgttggggtgagttgatgaatctggccacctgggtgggaagtaatttggaagacccagcatccagggaattagtagtaagctatgtcaatgttaatatgggcctaaaaatcagacaactattatggtttcacatttcctgtcttacttttggaagagaaactgttcttgagtatttggtgtcttttggagtgtggattcgcactcctcccgcttacagaccaccaaatgcccctatcttatcaacacttccggaaactactgttgttgccggcatgactgctgcccaaattaccgcaggtatcgcgcttcatcagtctaatctcaacggcagttctaaacttattcctaatgcttctctcatcgaaaactgtactaaggcggagttgagcggctctgcggagttgggggagtacgaaaagctactcaactcggtgcttgaacccatcggcagttctgaatatctcaacaaaatccaaaatagcctaagtacagagtggagtccttgtagcgtaacgagcggctctgtcagtcccttctatagctacatcaaaacaagtaatattctggcagatgacgtaggcagttctgctaaagccgtagcagcgtggacactaaaggctgccgcaagcggctccacttgtcctccctgcccagctccgagcggctctgaacactggtcttacgggctgaggcctggcagtggatccgagcattggtcttacggcctgagacctggcggcagttctgaacactggtcatacggactcagaccaggaagcggctctgagcattggtcttatggcctgcgccctggcggcagttctgaacactggtcatacggactcagaccaggaagcggctctgagcattggtcttatggcctgcgccctggcggcagttctgaacattggtcttacggactgaggcctggaagtggatccgagcactggtcttatggcctccgccctggcggcagttcttagtagtctaga

SEQ ID NO: 12 - protein, 683 aa, Artificial Sequence
  GnRH8-Hinge-Thelp-LFn-HBc-Thelp-hinge-GnRH8
MDAMKRGLCCVLLLCGAVFVSPSGTGSEHWSYGLRPGSGSEHWSYGLRPGGSSEHWSYGLRPGSGSEHWSYGLRPGGSSEHWSYGLRPGSGSEHWSYGLRPGGSSEHWSYGLRPGSGSEHWSYGLRPGTCPPCPAPKLIPNASLIENCTKAELGALNNRFQIKGVELKSQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLIDIDTYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVKLIPNASLIENCTKAELGALNNRFQIKGVELKSTCPPCPAPEHWSYGLRPGSGSEHWSYGLRPGGSSEHWSYGLRPGSGSEHWSYGLRPGGSSEHWSYGLRPGSGSEHWSYGLRPGGSSEHWSYGLRPGSGSEHWSYGLRPG

SEQ ID NO: 13 - DNA, 2326 bp, Artificial Sequence
  GnRH multimer-PA83 Antigen Cassette
aagcttccaccaaactcatcccaaacgcatcactcatcgagaactgcaccaaggctgaactggaggtcaaacaggagaataggctgctgaacgagagcgagagctcctctcagggcctgctcggatactatttttccgacctgaacttccaggctccaatggtggtcacaagttcaaccacaggggatctctcaatccccagctccgagctggaaaatattcctagcgagaaccagtactttcagtctgccatctggagtggcttcattaaggtgaagaaatctgacgagtatacctttgctacaagtgcagataatcacgtgaccatgtgggtcgacgatcaggaagtgatcaacaaggcctccaactctaataagattcgcctcgagaaaggtcgactgtaccagatcaaaattcagtatcagcgggagaaccctaccgaaaagggcctcgacttcaaactgtactggacagattctcagaataagaaagaagtgatctctagtgacaacctccagctgccagagctgaagcagaaatcaagcaattcccgaaagaaaaggagtacatcagccggccccactgtgcctgacagggataacgacggaatcccagactctctggaggtcgaagggtatactgtggatgtcaagaacaagagaacctttctgtcaccctggatcagcaacattcatgaaaagaaaggactgactaagtacaaatcctctccagagaagtggagtaccgcctcagatccctatagcgacttcgaaaaggtgacagggcggatcgacaaaaacgtcagccctgaggctcgacacccactggtggcagcttaccccattgtgcatgtcgatatggagaatatcattctgtccaagaacgaagaccagtctactcagaataccgatagtgagactcgaaccatctcaaagaacacaagcacttccaggacccacacatccgaggtgcatggaaacgctgaagtccacgcatctttctttgacatcggcggatctgtgagtgctgggttctcaaacagcaatagttcaaccgtcgcaattgatcattccctctctctggccggcgagaggacatgggctgaaactatgggcctcaacacagccgacactgctaggctgaacgcaaatatcagatacgtgaatactggaaccgccccaatctacaacgtgctgcccactacctccctcgtcctggggaagaatcagaccctggccacaatcaaggctaaagagaaccagctcagtcagattctggcccccaacaattactatccttctaagaatctcgcacctatcgccctgaacgctcaggacgattttagctccactccaattaccatgaactacaatcagttcctcgagctggaaaagaccaaacagctccggctggatacagaccaggtgtacggaaatatcgcaacatataactttgagaatggtcgggtgcgcgtcgacactggcagcaactggtccgaggtgctgcctcagatccaggaaacaactgcccgcatcattttcaatggcaaggatctcaacctggtggagaggagaatcgcagccgtcaacccaagcgaccccctggagaccacaaagcccgatatgaccctcaaggaagcactgaaaatcgccttcgggtttaatgagcctaacggtaatctgcagtaccagggcaaggacatcacagaattcgattttaacttcgaccagcagactagccagaacattaagaatcagctcgctgagctgaacgcaaccaatatctacacagtgctcgacaagatcaagctgaacgctaagatgaatatcctgattagagataaacggttccactatgaccgcaacaatatcgcagtgggtgccgatgaatccgtggtcaaggaggcacatcgagaagtcatcaattctagtaccgagggactgctcctgaacattgataaggacatcaggaaaattctgtctggatacatcgtggagattgaagacacagaggggctgaaggaagtcatcaatgatagatatgacatgctcaacatttcaagcctgcggcaggatggcaagacctttatcgacttcaagaagtacaacgataaactccctctgtacatcagcaacccaaattacaaggtgaacgtctatgctgtgactaaagagaacaccatcattaatcctagcgaaaacggagacacatccactaacgggatcaagaaaatcctgatttttagcaagaaaggttacgaaatcgggggtgccctcaacaatagattccagattaagggcgtggagctgaaaagctgtagtctaga

SEQ ID NO: 14 - protein, 1043 aa, Artificial Sequence
  GnRH multimer-PA83 Antigen Cassette
MDAMKRGLCCVLLLCGAVFVSPSGTGSEHWSYGLRPGSGSEHWSYGLRPGGSSEHWSYGLRPGSGSEHWSYGLRPGGSSEHWSYGLRPGSGSEHWSYGLRPGGSSEHWSYGLRPGSGSEHWSYGLRPGTCPPCPAPKLIPNASLIENCTKAELGALNNRFQIKGVELKSEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIGKLIPNASLIENCTKAELGALNNRFQIKGVELKSTCPPCPAPHWSYGLRPGSGSEHWSYGLRPGGSSEHWSYGLRPGSGSEHWSYGLRPGGSSEHWSYGLRPGSGSEHWSYGLRPGGSSEHWSYGLRPGSGSEHWSYGLRPG

SEQ ID NO: 15 - DNA, 1490 bp, Artificial Sequence
  GnRH multimer-leukotoxin Antigen Cassette
atggcaaccgtgattgacctctcattcccaaaaacaggggcaaaaaagattatcctctacatcccacagaactaccagtacgacactgagcagggcaacggactgcaggacctcgtgaaggccgctgaggaactggggatcgaagtccagagggaggaaagaaacaatattgcaactgcccagaccagtctgggcacaatccagactgcaattggcctgactgagcgaggaatcgtgctctctgctccccagattgataagctgctccagaagaccaaagcaggacaggctctgggaagcgcagagtccatcgtgcagaacgcaaataaggccaaaaccgtcctgtccgggatccagtctattctggggagtgtgctcgccggtatggacctggatgaggccctccagaacaatagcaaccagcacgctctggcaaaagccggactggagctcactaattctctgatcgaaaacattgccaatagtgtgaagaccctggacgagttcggggaacagatctcacagtttggtagcaaactgcagaacattaaggggctgggtaccctcggcgataagctgaaaaatatcggaggactcgacaaggcaggactgggactcgatgtgatctccggactgctctctggtgctaccgcagcactggtgctcgcagacaagaacgcttccacagcaaagaaagtcggggcaggtttcgagctggccaaccaggtggtcggcaatattacaaaggccgtgagctcctatatcctggctcagagagtcgctgcaggcctctctagtacaggacccgtggccgctctgattgcttctactgtcagtctggcaatctctcctctcgctttcgcaggaattgccgataagttcaaccacgctaagtcactggagagctacgccgaacggttcaagaaactggggtatgacggcgacaatctgctcgctgagtaccagcgcggcaccggaacaatcgacgcatccgtgaccgccattaacacagctctggcagcaatcgcaggaggtgtctctgctgcagcagctaatctgaaggatctcacatttgagaaggtgaaacataacctggtcatcactaatagcaagaaagaaaaagtgaccattcagaactggttcggcgaggccgactttgctaaggaagtgccaaattataaggccactaaagatgagaagatcgaggaaatcattggacagaacggggaaggtatcaccagcaaacaggtggacgatctgattgctaaaggcaacggaaagatcacacaggacgagctgagcaaggtggtcgataattacgaactgctcaaacatagcaagaacgtgacaaattccctggacaagctcatctcaagcgtctcagccttcacatcctctaacgacagcggaaatgtgctggtcgctcccactagtatgctcgatcagtcactgagttactccagtttgccagggggtcctagtagtctaga

SEQ ID NO: 16 - protein, 738 aa, Artificial Sequence
  GnRH multimer-leukotoxin Antigen Cassette
MDAMKRGLCCVLLLCGAVFVSPSGTGSEHWSYGLRPGSGSEHWSYGLRPGGSSEHWSYGLRPGSGSEHWSYGLRPGGSSEHWSYGLRPGSGSEHWSYGLRPGGSSEHWSYGLRPGSGSEHWSYGLRPGTCPPCPAPMATVIDLSFPKTGAKKIILYIPQNYQNDTEQGNGLQDLVKAAEELGIEVQREERNNIATAQTSLGTIQTAIGLTERGIVLSAPQIDKLLQKTKAGQALGSAESIVQNANKAKTVLSGIQSILGSVLAGMDLDEALQNNSNQHALAKAGLELTNSLIENIANSVKTLDEFGEQISQFGSKLQNIKGLGTLGDKLKNIGGLDKAGLGLDVISGLLSGATAALVLADKNASTAKKVGAGFELANQVVGNITKAVSSYILAQRVAAGLSSTGPVAALIASTVSLAISPLAFAGIADKFNHAKSLESYAERFKKLGYDGDNLLAEYQRGTGTIDASVTAINTALAAIAGGVSAAAANLKDLTFEKVKHNLVITNSKKEKVTIQNWFGEADFAKEVPNYKATKDEKIEEIIGQNGEGITSKQVDDLIAKGNGKITQDELSKVVDNYELLKHSKNVTNSLDKLISSVSAFTSSNDSGNVLVAPTSMLDQSLSSLQFARGSTCPPCPAPEHWSYGLRPGSGSEHWSYGLRPGGSSEHWSYGLRPGSGSEHWSYGLRPGGSSEHWSYGLRPGSGSEHWSYGLRPGGSSEHWSYGLRPGSGSEHWSYGLRPG

SEQ ID NO: 17 - protein, 10 aa, Unknown
  GnRH Sequence
EHWSYGLRPG

SEQ ID NO: 18 - protein, 8 aa, Artificial Sequence
  Synthetic peptide
MATVIDLS

SEQ ID NO: 19 - protein, 534 aa, Artificial Sequence
  Synthetic polypeptide
MDAMKRGLCCVLLLCGAVFVSPSGTGSEHWSYGLRPGSGSEHWSYGLRPGGSSEHWSYGLRPGSGSEHWSYGLRPGGSSEHWSYGLRPGSGSEHWSYGLRPGGSSEHWSYGLRPGSGSEHWSYGLRPGTCPPCPAPKLIPNASLIENCTKAELGALNNRFQIKGVELKSQEEHLKEIMKHIVKIEVKGEEAVKKEAAEKLLEKVPSDVLEMYKAIGGKIYIVDGDITKHISLEALSEDKKKIKDIYGKDALLHEHYVYAKEGYEPVLVIQSSEDYVENTEKALNVYYEIGKILSRDILSKINQPYQKFLDVLNTIKNASDSDGQDLLFTNQLKEHPTDFSVEFLEQNSNEVQEVFAKAFAYYIEPQHRDVLQLYAPEAFNYMDKFNEQEINLKLIPNASLIENCTKAELGALNNRFQIKGVELKSTCPPCPAPEHWSYGLRPGSGSEHWSYGLRPGGSSEHWSYGLRPGSGSEHWSYGLRPGGSSEHWSYGLRPGSGSEHWSYGLRPGGSSEHWSYGLRPGSGSEHWSYGLRPG

(19) United States
(12) Patent Application Publication
(10) Pub. No.: US 2023/0136261 A1
(43) Pub. Date: May 4, 2023

(54) METHODS AND COMPOSITIONS FOR VIRAL VECTORED GnRH VACCINES TO CONTROL REPRODUCTION AND BREEDING BEHAVIOR IN MAMMALS

(71) Applicants: Henry BAKER, Auburn, AL (US); Jianfeng ZHANG, Rockville, MD (US); Kent Rigby VAN KAMPEN, Payson, UT (US); Scot ROBERTS, Gaitherburg, MD (US); De-chu Christopher TANG, Gaitherburg, MD (US); AUBURN UNIVERSITY, AUBURN, AL (US); ALTIMMUNE INC., GAITHERSBURG, MD (US)

(72) Inventors: Henry BAKER, Auburn, AL (US); Jianfeng ZHANG, Rockville, MD (US); Kent VAN KAMPEN, Payson, UT (US); Scot ROBERTS, Frederick, MD (US); De-chu TANG, Gaithersburg, MD (US)

(21) Appl. No.: 16/347,573

(22) PCT Filed: Nov. 9, 2017
(86) PCT No.: PCT/US17/60792

§ 371 Date: May 4, 2019

Related U.S. Application Data
(60) Provisional application No. 62/419,507, filed on Nov. 9, 2016.

(51) Int. Cl. A61K 39/00; C12N 15/861; A61K 39/385

(52) U.S. Cl. CPC A61K 39/0006; C12N 15/861; A61K 39/385; C12N 2710/10041; A61K 2039/552; A61K 2039/5256; A61K 2039/6037

(57) ABSTRACT

Immunogenic compositions comprising a recombinant adenoviral vector that expresses a nucleic acid molecule encoding multimers of a Gonadotrophic Releasing Hormone (GnRH), an antigenic carrier and multiple immune enhancing epitopes are described herein. The use of the immunogenic compositions on mammals resulting in an antibody response to GnRH can inhibit the physiological activity of GnRH and thus induce infertility and modify breeding behavior of immunized animals.

### CROSS-REFERENCE TO RELATED APPLICATIONS

This application is a national stage filing under 35 U.S.C. 371 of International Application No. PCT/US17/60792, filed Nov. 9, 2017, entitled METHODS AND COMPOSITIONS FOR VIRAL VECTORED GnRH VACCINES TO CONTROL REPRODUCTION AND BREEDING BEHAVIOR IN MAMMALS, which claims benefit of Provisional Patent application U.S. Ser. No. 62/419,507 filed Nov. 9, 2016, and the entire contents of all priority applications are incorporated herein by reference.

### SEQUENCE LISTING

This application hereby incorporates by reference the material of the electronic Sequencing Listing filed concurrently herewith. The materials in the electronic Sequence Listing is submitted as a text (.txt) file entitled “ALT2025.US1_Seglist.txt” created on Nov. 8, 2019, which has a file size of 44 KB, and is herein incorporated by reference in its entirety.

### FIELD OF THE INVENTION

The disclosure relates to methods and compositions for inducing infertility and controlling breeding behavior in a mammal by providing an adenovirus (Ad) vectored vaccine against endogenous gonadotropin releasing hormone (GnRH).

### BACKGROUND OF THE INVENTION

There are several noteworthy reasons for utilizing recombinant Ad vector as a vaccine carrier. These include 1) Ad vectors are capable of transducing both mitotic and postmitotic cells in situ (Shi 1999); 2) stocks containing high titers of virus (greater than 10¹² pfu (plaque forming unit) per ml)) can be prepared, making it possible to transduce cells in situ at high multiplicity of infection (MOI); 3) the vector is safe based on its long-term use as a vaccine; 4) the virus is capable of inducing high levels of transgene expression (at least as an initial burst) and 5) the vector can be engineered to a great extent with versatility. Recombinant Ad vectors have been utilized as vaccine carriers by intranasal, epicutaneous, intratracheal, intraperitoneal, intravenous, subcutaneous and intramuscular routes.

Ad-vectored nasal vaccine appears to be more effective in eliciting an immune response than injection of DNA or topical application of Ad (Shi et al. (2001) J. Virol. 75:11474-11482). Previously reported results have shown that the potency of the E1/E3 defective Ad5 vector as a nasal vaccine carrier is not suppressed by a preexisting immunity to adenovirus (Xiang et al. (1996) Virology 219(1) 220-7; Shi et al. 2001).

Ad-based vaccines mimic the effects of natural infections in their ability to induce major histocompatibility complex (MHC) class I restricted T-cell responses yet eliminate the possibility of reversion back to virulence because only a subfragment of the pathogen's genome is expressed from the vector. This “selective expression” may solve the problem of differentiating vaccinated-but-uninfected animals from their infected counterparts, because the specific markers of the pathogen not encoded by the vector can be used to discriminate the two events. Notably, propagation of the pathogen is not required for generating vectored vaccines because the relevant antigen genes can be amplified and cloned directly from field samples (Rajakumar et al., 1990). This is particularly important for production of highly virulent influenza strains, such as H5N1, because this strain is too dangerous and difficult to propagate (Wood et al., 2002).

Uncontrolled growth of wild and domestic animal populations is an international problem of epidemic proportions and the frequency and danger of wild animal and human conflicts are rising at an alarming rate. The all too familiar statistics of damage done by deer illustrates the magnitude of this problem. Each year 1.5 million car accidents result from collisions with deer, costing 150 human lives, 10,000 human injuries and $1 billion in vehicle damage. There are many other examples of damage done to crops by wild pigs, loss of pets and livestock by coyotes, bite wounds caused by unwanted dogs and cats and diseases transmitted to domestic livestock and people. Killing these harmful animals attacks the result not the cause of this problem and despite the danger, is distasteful to the public. The cause is uncontrolled breeding and the only sound solution is to prevent growth of these animal populations by preventing reproduction.

The suffering created by abandonment, abuse, mass killing and the hardships endured by homeless dog and cat strays probably constitutes the single greatest source of cruelty to two of our most endeared pet species. Additionally, every community must support animal control units and shelters at an enormous financial burden. It is estimated that animal control and humane organizations spend $250 to capture, process, adopt or euthanize each dog or cat. This amounts to a national expenditure of $2.5 to 3.75 billion each year. Sincere attempts have been made to stem this tide, but the problem continues unabated and will continue until an effective non-surgical contraceptive is developed for dogs and cats. The ideal contraceptive for animal control must be simple to administer, requiring no more than an injection, topical, intranasal or oral administration, capable of being given rapidly to large numbers of animals, effective in preventing conception and reproductive behavior, safe, inexpensive, and ideally linked with infectious disease control antigens such rabies immunization, which is readily accepted by animal owners and control agencies.

For at least 40 years scientists have been developing contraceptive vaccines for use in humans, and substantial progress has been made, leading to clinical development (Talwar and Gaur, 1987; Alexander and Bialy, 1994). The goals of human contraceptive vaccines are substantially different and more difficult to achieve than an ideal animal vaccine, and include requirements such as reversibility, no modification of reproductive behavior, and no detectable changes in the tissues of reproductive organs. Although the specific objectives of immunocontraception are very different between humans and animals, the basic techniques are similar.

One major impediment of immunocontraception is to trick the body into mounting an immune response against itself, in the form of hormones or structural components of eggs and sperm. This is much more problematic than designing vaccines for foreign antigens, such as infectious organisms. Other technical limitations of conventional protein based immunization include the need to highly purify compounds (e.g., hormones) which normally exist in very small quantities in the body, the difficulty in producing enough of these purified proteins to immunize an animal, let alone thousands or millions of animals, the problems of maintaining these temperature sensitive materials from manufacture to the point of use to assure their potency and effectiveness, and the obvious high cost of overcoming these problems.

Three methods are currently favored to achieve successful immunocontraception: induction of immunity against reproductive hormones, immunization against sperm antigens and immunity to the zona pellucida, a protein corona which surrounds the egg and facilitates fertilization by sperm. All three approaches have advantages and limitations to achieve the essential characteristics of a useful animal control contraceptive vaccine including: (a) prevention of fertility, (b) elimination of reproductive behavior such as prevention of the female going into “heat”, (c) long term effectiveness, in some cases preferably permanent (d) efficiencies exceeding 60% after initial immunization and higher after multiple vaccinations, (e) inexpensive to manufacture, (t) stable under field conditions, (g) easy to administer, and (h) free from serious non-reproductive health consequences. Zona pellucida is a target since antibodies to this protein interfere directly with fertilization and it is highly immunogenic across some but not all species (e.g., porcine ZP3 is immunogenic for horses, but not dogs or cats). It has been shown that the native protein derived from swine is antigenic in cats, but does not interfere with fertility, presumably because the antigenic epitopes to which cats respond have no corresponding sites on the native feline zona pellucida (Gorman et al., 2002). This vaccine has several serious limitations. For example, it affects only females and does not alter reproductive behavior. Finally, to date only native proteins derived principally from pig ovary zona pellucida has been used as an immunogen, because recombinant protein derived from the native cDNA sequence lacks the post translational modification necessary for an antibody to interfere with native zona pellucida function. For these reasons, the anti-zona pellucida vaccines are of limited value for immunocontraception for most animal control situations.

Gonadotrophin-releasing hormone (GnRH) is a decapeptide trophic hormone required for normal reproduction in both males and females. Therefore, GnRH-specific immunization, can be used for both sexes (Fraser et al., 1974; Clark et al., 1978; Silversides et al., 1990; Jeffcoate et al., 1974; Hsu et al., 2000). Treatments that inhibit GnRH function would also suppress reproductive behavior. GnRH is highly conserved in all mammals and most other vertebrates. Therefore, anti-GnRH vaccination methods are effective across all mammals of interest for population control. Because GnRH is a very small decapeptide and is recognized by the body as self, it presents a challenge to induce immunity. To circumvent this problem, GnRH can be linked to a variety of antigenic carriers to enhance its immunological recognition, immune response and interference with normal function. Moreover, antigenicity can be increased by altering the number of GnRH repeats, with some evidence that the longer the GnRH multimer, the greater the antibody response.

However, there still remains a need for contraceptive vaccine that will increase the immunogenicity to the “self” GnRH epitopes with long term efficacy that can be achieved with a single dose or a prime-boost regimen. The adenoviral vectored contraceptive vaccines provided in this disclosure incorporate the latest advances in molecular biology and are equally effective in any animal species and either gender. That feature makes this product effective in controlling populations of deer, coyotes, horses, dogs, cats, pigs, etc without modification for each species. Additionally, a controlled release delivery system can be devised.

Citation or identification of any document in this application is not an admission that such document is available as prior art to the present invention.

### SUMMARY OF THE INVENTION

In certain embodiments are provided an immunogenic composition which may comprise an adenovirus vector that contains and expresses a nucleic acid encoding a recombinant protein, which may comprise a carrier immunogenic antigen, and mammalian GnRH or homolog thereof. Herein referred to as an Ad-vectored GnRH vaccine or construct. GnRH is a short peptide and to increase the immunogenicity of the expressed peptide and provide more possible epitopes for antibody generation, repeats or immunogenic monomers of the GnRH sequence, optionally attached by a linker, may be used. The GnRH sequence may be represented about 6 to about 20 times to create a multimer of GnRH, which may be present before or after, or both, of the carrier antigen(s). In embodiments, the antigen is flanked by about 6 to 10 linear repeats of GnRH. In certain embodiments, the adenovirus vector further comprises one or more T cell epitopes, wherein those T cells epitopes are expressed in the recombinant protein.

In embodiments, the carrier antigen comprises a bacterial or viral immunogenic antigen, or immunogenic fragment thereof. In certain embodiments, the carrier antigen comprises leukotoxin antigen, B. anthracis lethal factor, B. anthracis protective antigen, tetanus toxin, diphtheria toxin, Hepatitis B core antigen, or a combination thereof. In embodiments, the B. anthracis antigen is lethal factor, protective antigen, including PA83, PA63, or immunological fragments thereof. The adenovirus vector may be selected from an E1, E3, and/or E4 deleted or disrupted adenovirus. In embodiments, the adenovirus vector is replication deficient.

In certain embodiments provided herein is an immunogenic composition comprising: an adenovirus vector that contains and expresses a nucleic acid encoding a recombinant protein, comprising a leukotoxin antigen, and endogenous mammalian GnRH or homolog thereof. In embodiments, the GnRH sequence may be represented about 6 to about 20 times to create a multimer of GnRH, which may be present before or after, or both, of the leukotoxin antigen. In embodiments, the antigen is flanked by about 6 to 10 linear repeats of GnRH. In certain embodiments, the adenovirus vector further comprises one or more T cell epitopes, wherein those T cells epitopes are expressed in the recombinant protein.

In embodiments, the adenovirus vector further comprises (in addition to the leukotoxin antigen), and the expressed recombinant protein comprises, a carrier antigen comprising a bacterial or viral immunogenic antigen, or immunogenic fragment thereof. In certain embodiments, the carrier antigen comprises B. anthracis lethal factor, B. anthracis protective antigen, tetanus toxin, diphtheria toxin, Hepatitis B core antigen, or a combination thereof. In embodiments B. anthracis protective antigen is PA83, PA63 or an immunogenic fragment thereof.

In embodiments are provided immunogenic formulations for administration to a mammal which may comprise the present GnRH Ad-vectored vaccine and optionally an adjuvant. The formulation may be a liquid, a solid, lyophilized, or a suspension and the optional adjuvant may be CpG, GMCSF, a TLR3 agonist or other adjuvants. In embodiments, the formulation may further comprise a delivery system or device.

In embodiments provided herein are methods for inducing an immune response against GnRH in a mammal, wherein the GnRH Ad-vectored vaccine is administered to the mammal. Administration includes intradermal, subcutaneous, intramuscular, intravenous, oral, topical or intranasal. The mammal may be a companion animal, a domesticated animal, a feral animal, a food-or feed-producing animal, a livestock animal, a game animal, a racing animal, a performance animal, or a sport animal. In particular, the mammal is a bovine, e.g., cow, equine, e.g., horse, canine, e.g., dog, feline, e.g., cat, a caprine, e.g., goat, ovine, e.g., sheep, porcine, e.g., pig, other ungulate e.g., deer, or any other mammal. In certain embodiments, the immune response against GnRH comprises inducing anti-GnRH antibody production. In certain other embodiments, the immune response against GnRH induces infertility. In embodiments, the infertility of the mammal is sustained for a period of at least about 1 to 12 months, about 2 to 4 years or at least 4 years after the initial administration of the immunogenic composition, and optionally for the length of the animal's life.

In certain embodiment, the methods of inducing an immune response against GnRH comprises a homologous prime-boost dosing regimen or a heterologous prime-boost dosing regimen. In embodiments, the heterologous boost dose comprises a GnRH carrier protein construct. In exemplary embodiments, the heterologous boost dose comprises GnRH-diphtheria toxoid construct.

In embodiments provided herein are methods for inducing infertility in a mammal, comprising, wherein a prime dose comprising the GnRH Ad-vectored vaccine is administered to the mammal followed by administration of a heterologous boost dose comprising a GnRH-protein construct. In embodiments, the heterologous boost dose is administered about 4 weeks to about 52 weeks after administration of the prime dose. In certain embodiments, the method further comprises administering a homologous boost dose before administering the heterologous boost dose to the mammal. In embodiments, the infertility of the mammal is sustained for a period of at least about 1 to 12 months, about 2 to 4 years or at least 4 years after the prime dose of the Ad-vectored GnRH vaccine, and optionally for the length of the animal's life.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 shows an adenovirus vector expressing GnRH antigen, including any of the present GnRH constructs comprising repeats, carrier antigen, linker sequences, hinge sequences, T cell epitope sequences, disclosed herein.

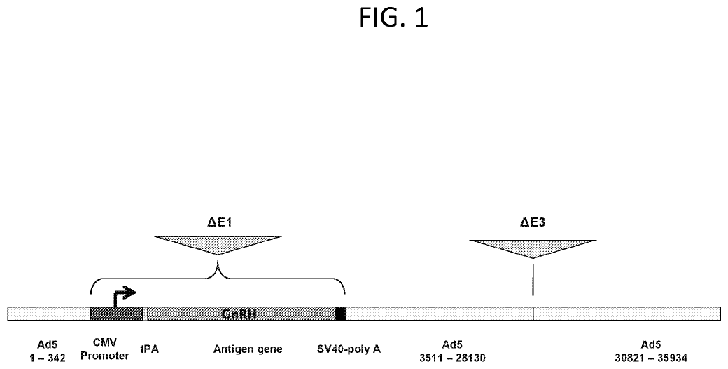

FIG. 2 shows fold increase over control (baseline) of anti-GnRH antibody titer in male mice at given time points following a single vaccination using 1×10⁹ ifu of five Ad-vectored GnRH vaccines tested. Each bar represents an Ad-GnRH vaccine engineered to express 16 multimers of GnRH linked to one of five antigenic protein carriers (LKT (leukotoxin A1)-GnRH16; LTK-GnRH16Dog (codon optimized); B. Anthracis PA83 (protective antigen)-GnRH16; B. anthracis LF (lethal factor)-GnRH16; and, LF-Hepatitis B core antigen (HbC)-GnRH). The highest antibody titer at all time points was produced in response to vaccination utilizing the bacterial leukotoxin (leukotoxin A1 gene of Pasteurella haemolytica) carrier protein.

FIG. 3 shows testosterone values for immunized mice of combined Ad-vectored GnRH vaccine #8 and #9 which express the same antigen, AdLKTGnRH16 and differ only in their species codon optimization, and adjuvant (CGMCSF or CPG). The data suggests that values below 1 ng/ml may be a threshold for fertility. Data points at or below that threshold are observed at 30 days after a prime immunization and decrease with time.

FIG. 4 shows testicular volumes, expressed as percent of age matched controls, compared with days after primary immunization for immunized mice of combined Ad-vectored GnRH vaccine #8 and #9 which express the same antigen, AdLKTGnRH16 and differ only in their species codon optimization, and adjuvant (CGMCSF or CPG). The number of mice with testicular volumes at or below 60% of controls increased with time. The histopathological observations indicate that mice with testicular volumes less than 30% of controls are functionally emasculated.

FIG. 5 shows a correlation between low testosterone and testicular volume for individual animals with the five Ad-vectored GnRH vaccines tested at 90 days post-prime immunization, showing positive correlation between serum testosterone levels and testicular volume that is strongest for vaccines Ad-Leukotoxin-GnRH16Mouse (B) and Ad-Leukotoxin-GnRH16Dog (C) and Ad-LF-HBc-GnRH16 (F), but not for controls (A).

FIG. 6 shows histopathological changes in dysgenic testicles of immunized mice compared with normal testicular structure and spermiogenesis of CD1 mice. A: Normal testicular histological structure showing well defined Leydig cells and spermiogenesis. B: Testicular dysgenesis showing loss of Leydig cells and aspermiogenesis in mice from Ad-vectored GnRH vaccine #9, 90 days post-primary immunization (PI). C: Testicular dysgenesis showing loss of Leydig cells and aspermiogenesis (vaccine #11, 60 days PI). D: Testicular dysgenesis showing loss of Leydig cells, intertubular inflammation and aspermiogenesis (vaccine #12, 60 days PI). Magnification for all figures 25.2×.

FIG. 7 shows gross necropsy appearance of a normal size testicle (left) compared with profound dysgenesis of an immunized mouse testicle.

FIG. 8 shows serum anti-GnRH antibody production following homologous Ad-GnRH prime-boost (Wks. 0-48) and after heterologous boost (Wks. 48-70). Arrows at 0 and about week 4 indicate immunization with Ad-GnRH, arrow at about week 49 indicates immunization with low dose GnRH protein.

FIG. 9 shows progesterone and anti-GnRH antibody production following a homologous administration protocol study in mares using Ad-GnRH prime-boost (Phase One (Week 0-46): Ad-GnRH prime day 0, Ad-GnRH boost week 4). Arrows represent days the Ad-GnRH construct was administered to the mares. See Example 5 for details of the construct.

FIG. 10 shows progesterone and anti-GnRH antibody production following a heterologous prime boost administration protocol study in mares using Ad-GnRH prime-boost (Phase Two (Week 0-46): Ad-GnRH prime day 0, protein antigen boost at week 49). Arrow represent week the protein antigen boost was administered to the mares. See Example 5 for details of the Ad-GnRH construct.

FIG. 11 shows frequency distribution of anti-GnRH antibody data for treatment mares during phase two (Ad-GnRH prime day 0, protein antigen boost at week 49). Following the protein antigen boost at week 49 an increase in frequency distribution is shown. Arrow represent week the protein antigen boost was administered to the mares.

### DETAILED DESCRIPTION OF THE INVENTION

The present invention provides methods and compositions for inducing an immune response in a mammal against host GnRH following administration of an adenoviral vectored (Ad-vectored) vaccine expressing a recombinant protein which may comprise GnRH peptide sequence(s) and a heterogeneous (e.g. non-mammalian antigen) immunogenic carrier antigen. The use of an Ad-vector in combination with strategic placement of GnRH sequences (a 10 amino acid peptide) providing multimers of GnRH and heterogeneous immunogenic carrier antigens such bacterial or viral antigens (e.g. tetanus toxin, diphtheria toxin, B. anthraces lethal factor or protective antigen, and/or Hepatitis B core antigen) result in a robust autoimmune response against host GnRH. As used herein “endogenous” refers to the naturally expressed GnRH of the host mammal; endogenous and natural GnRH are herein used interchangeably. As used herein “heterogenous” refers to a non-host, non-mammalian antigen. Exemplary heterogenous carrier antigens are bacterial or viral antigens.

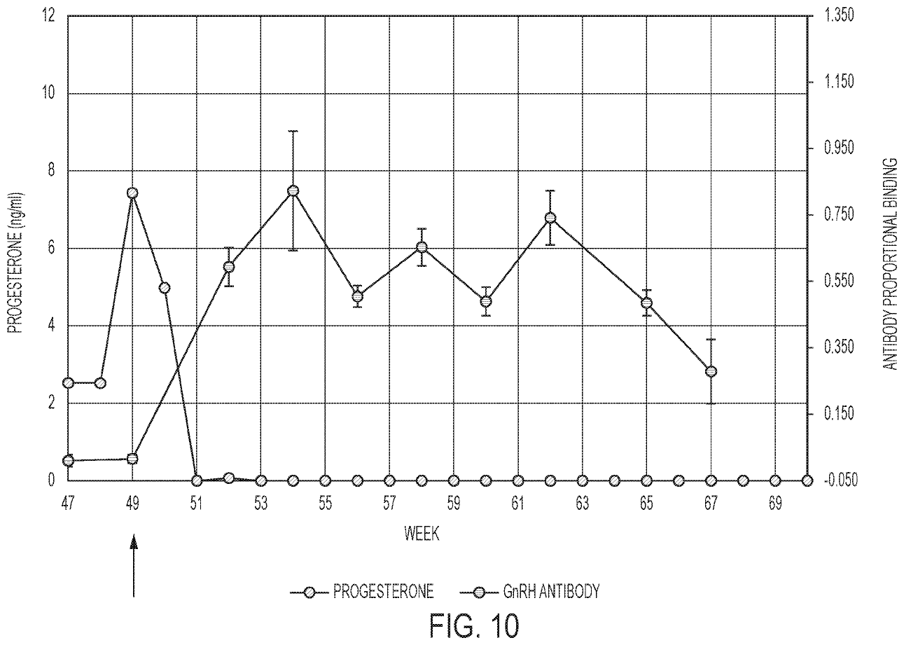

An immune response against GnRH, an apex hormone in the fertility cascade, results in not only infertility (immune-castration and/or immune-contraceptive) but alters breeding behavior. That improvement in the immunogenicity of the GnRH epitopes provides for a practical vaccine for animals, such as wild, feral or range animals, that may only need to be administered once to induce infertility for the length of the animal's life. However, Applicants found that a heterologous dosing regimen (e.g. Ad-GnRH construct prime dose following by a GnRH protein boost) in mares resulted in a robust method of inducing infertility in those mares. See Example 5 and FIG. 10.

GnRH is highly conserved across many mammals, pyroGlu-His-Trp-Ser-Tyr-Gly-Leu-Arg-Pro-Gly-NH2 (SEQ ID NO: 1) is the endogenous or natural hormone for all mammals such as mice, cats, dogs, horses, pigs, deer, etc., and therefore the same Ad-vectored GnRH vaccine, with optional codon optimization, can be used to the same effect across various mammalian species. The expressed GnRH monomer subunits may or may not contain terminal modifications of the natural peptide, e.g. pyroglutamic acid at the N-terminal end and carboxamide at the C-terminal glycine.

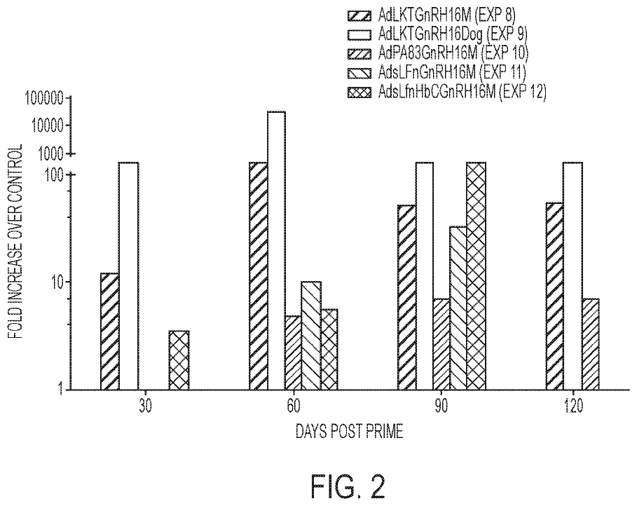

In embodiments, the Ad-vectored GnRH vaccine (herein referred to as an “immunogenic composition” or “immunogenic formulation” containing that composition), when administered to mammals induces antibody production against GnRH that correlates with a reduction in testosterone or progesterone levels indicating the GnRH antibodies are able to neutralize host GnRH, blocking the effects of that hormone. See FIGS. 2, 3 and 10. The induced anti-GnRH antibodies neutralize circulating GnRH, thereby removing the stimulus for production and release of the gonadotropic hormones—luteinising hormone (LH) and follicle stimulating hormone (FSH)—from the pituitary gland. LH and FSH are both required for the development and maintenance of ovarian function, wherein the effect of vaccination with the present Ad-GnRH vaccine is to cause suppression of ovarian activity. Production of the female sex hormones progesterone and oestradiol is significantly reduced, leading to a reduction in oestrus-related behavior in mammals.

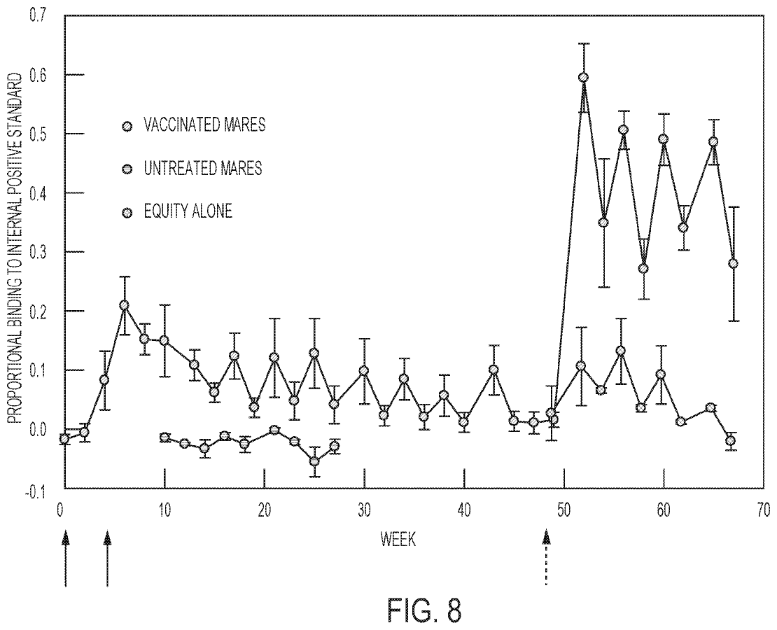

In embodiments, the Ad-vectored GnRH vaccine, when administered to normal estrous cycling mares induces antibody production against GnRH that correlates with suppressed estrous cyclicity and a reduction in breeding behaviour. See Example 4, 5; and, FIGS. 8 and 10. In embodiments, use of the present Ad-vectored GnRH vaccine induces a state of ‘immune anoestrus’—a physiological state that mimics seasonal (winter) anoestrus in fillies and mares.

In embodiments, use of the Ad-vectored GnRH vaccine employs homologous prime-boost doing regimen, wherein the boost dose of the Ad-vectored GnRH vaccine is administered days, weeks or months after the initial prime dose. In embodiments, the Ad-vectored GnRH vaccine is administered at least once, at least twice, at least three times or more to a mammal wherein an immune response against host GnRH is induced. In embodiments, the immune response comprises an antibody response against GnRH.

In alternative embodiments, use of the Ad-vectored GnRH vaccine to induce an immune response against GnRH employs a heterologous prime-boost dose regimen, wherein the heterologous boost dose is administered days, weeks or months following the initial prime dose. In exemplary embodiments, the heterologous boost dose comprises GnRH such as a GnRH linked to a carrier protein (e.g. GnRH-protein construct), a DNA vector expressing GnRH including bacterial or viral vectors. In embodiments, the heterologous boost dose comprises a vector expressing GnRH and optionally a carrier protein that is other than the present Ad-vectored GnRH vaccine. In alternative embodiments, the heterologous boost dose comprises the present Ad-vectored GnRH vaccine. The heterologous boost dose may further comprise an adjuvant.

In embodiments, the prime dose of a heterologous prime-boost dosing regimen comprises the present Ad-vectored GnRH vaccine. In alternative embodiments, the prime dose of a heterologous prime-boost dosing regimen comprises GnRH such as a GnRH linked to a carrier protein (e.g. GnRH-protein construct), a DNA vector expressing GnRH including bacterial or viral vectors. In embodiments, the heterologous prime dose comprises a vector expressing GnRH and optionally a carrier protein that is other than the present Ad-vectored GnRH vaccine. The heterologous prime dose may further comprise an adjuvant.

In embodiments, at least one prime dose and at least one boost dose in a heterologous prime-boost dosing regimen are administered to a mammal to induce an immune response against GnRH. In embodiments, at least two prime doses and at least one boost dose in a heterologous prime-boost dosing regimen are administered to a mammal to induce an immune response against GnRH. In embodiments, at least one prime dose and at least two boost doses in a heterologous prime-boost dosing regimen are administered to a mammal to induce an immune response against GnRH. As used herein, the prime dose and boost dose when employed in a heterologous prime-boost dosing regimen are different compositions temporally administered. In embodiments, the prime dose and boost dose employed in the heterologous prime-boost dosing regimen comprise GnRH or a vector (e.g., DNA plasmid, bacterial vector, viral vector, insect vector, etc.) expressing GnRH wherein at least one of the prime dose or boost dose comprise the present Ad-vectored GnRH vaccine.

As used herein an Ad-vectored GnRH vaccine may comprise and expresses a recombinant protein of interest that may comprise a B. anthraces antigen, GnRH peptide sequence(s) and optionally a Hep B core antigen and/or T cell epitopes. The instant disclosure provides a significant improvement in the effectiveness, including inducing long term infertility, with the use of an Ad-vectored GnRH vaccine for providing an immune response against endogenous/natural/host GnRH wherein the vaccine may be administered non-invasively or via an injection.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, the term, “adjuvant” refers to a pharmacological or immunological agent that modifies the effect of another agent, such as enhancing the immune response to a supplied antigen from a vaccine.

The terms “Ad-vector GnRH vaccine” or “Ad-vectored GnRH vaccine” as used herein interchangeably, refers to an adenoviral vector “immunogenic composition” that encodes immunogenic GnRH peptide sequence(s) and a heterogeneous “carrier” antigen(s). The immunogenic GnRH peptide(s) and heterogeneous “carrier” antigen(s) is expressed as a recombinant protein that induces anti-GnRH antibody generation. The adenovirus may be any adenovirus, such as but not limited to, a human adenovirus, a bovine adenovirus, a canine adenovirus, a non-human primate adenovirus, a chicken adenovirus, or a porcine or swine adenovirus.

As used herein, the term “human adenovirus” is intended to encompass all human adenoviruses of the Adenoviridae family, which include members of the Mastadenovirus genera. To date, over fifty-one human serotypes of adenoviruses have been identified (see, e.g., Fields et al., Virology 2, Ch. 67 (3d ed., Lippincott-Raven Publishers)). The adenovirus may be of serogroup A, B, C, D, E, or F. The human adenovirus may be a serotype 1 (Ad 1), serotype 2 (Ad2), serotype 3 (Ad3), serotype 4 (Ad4), serotype 5 (Ad5), serotype 6 (Ad6), serotype 7 (Ad7), serotype 8 (Ad8), serotype 9 (Ad9), serotype 10 (Ad10), serotype 11 (Ad11), serotype 12 (Ad12), serotype 13 (Ad13), serotype 14 (Ad14), serotype 15 (Ad15), serotype 16 (Ad16), serotype 17 (Ad17), serotype 18 (Ad18), serotype 19 (Ad19), serotype 19a (Ad19a), serotype 19p (Ad19p), serotype 20 (Ad20), serotype 21 (Ad21), serotype 22 (Ad22), serotype 23 (Ad23), serotype 24 (Ad24), serotype 25 (Ad25), serotype 26 (Ad26), serotype 27 (Ad27), serotype 28 (Ad28), serotype 29 (Ad29), serotype 30 (Ad30), serotype 31 (Ad31), serotype 32 (Ad32), serotype 33 (Ad33), serotype 34 (Ad34), serotype 35 (Ad35), serotype 36 (Ad36), serotype 37 (Ad37), serotype 38 (Ad38), serotype 39 (Ad39), serotype 40 (Ad40), serotype 41 (Ad41), serotype 42 (Ad42), serotype 43 (Ad43), serotype 44 (Ad44), serotype 45 (Ad45), serotype 46 (Ad46), serotype 47 (Ad47), serotype 48 (Ad48), serotype 49 (Ad49), serotype 50 (Ad50), serotype 51 (Ad51), or combinations thereof, but are not limited to these examples. In certain embodiments, the adenovirus is serotype 5 (Ad5).

As used herein “GnRH” (Gonadotropin-releasing hormone) refers to the natural tropic peptide hormone synthesized and released from GnRH neurons located in hypothalamus. GnRH is responsible for the release of follicle-stimulating hormone (FSH) and luteinizing hormone (LH) from the anterior pituitary. GnRH controls hypothalamic-pituitary-gonadal axis that stimulates the development and maintains function of the gonads. Gonadotropin-releasing hormone (GnRH), is also known as luteinizing hormone-releasing hormone (LHRH) and gonadoliberin. As used herein “GnRH” peptide present in the Ad-vectored GnRH vaccine may be any peptide epitope that will result in antibody generation against endogenous or host GnRH.

As used herein “host GnRH” refers to the natural or endogenous GnRH produced by the host mammal, wherein the host mammal is administered the Ad-vectored GnRH vaccine and may be selected from a companion animal, a domesticated animal, a feral animal, a food-or feed-producing animal, a livestock animal, a game animal, a racing animal, a performance animal, or a sport animal.

### Adenoviral GnRH (Ad-GnRH) Vectors and Compositions

The immunogenic compositions of interest may comprise an adenovirus vector (Ad-vector) that contains and expresses a nucleic acid encoding a recombinant protein, which may comprise a carrier immunogenic antigen(s), and mammalian GnRH sequence(s) or homolog(s) thereof. The immunogenic compositions are administered to a mammal wherein those compositions induce an immune response against host GnRH, e.g. an antibody response against GnRH. That immune response induces infertility, cessation of breeding behavior and may be used as therapy for gonadal proliferative diseases and/or cancer (e.g. prostate cancer).

Any adenoviral vector (Ad-vector) known to one of skill in art, and prepared for administration to a mammal, which may comprise and express an immunogenic antigen may be used with the methods of this application. Such Ad-vectors include any of those in U.S. Pat. Nos. 6,706,693; 6,716,823; 6,348,450; or US Patent Publ. Nos. 2003/0045492; 2004/0009936; 2005/0271689; 2007/0178115; 2012/0276138 (herein incorporated by reference in entirety).

In certain embodiments the recombinant adenovirus vector may be non-replicating or replication-deficient requiring complementing E1 activity for replication. In embodiments the recombinant adenovirus vector may include E1-defective, E3-defective, and/or E4-defective adenovirus vectors, or the “gutless” adenovirus vector in which viral genes are deleted. The E1 mutation raises the safety margin of the vector because E1-defective adenovirus mutants are replication incompetent in non-permissive cells. The E3 mutation enhances the immunogenicity of the antigen by disrupting the mechanism whereby adenovirus down-regulates MHC class I molecules. The E4 mutation reduces the immunogenicity of the adenovirus vector by suppressing the late gene expression, thus may allow repeated re-vaccination utilizing the same vector. In embodiments, the recombinant adenovirus vector is an E1 and/or E3 defective vector.

The “gutless” adenovirus vector replication requires a helper virus and a special human 293 cell line expressing both Ela and Cre, a condition that does not exist in natural environment; the vector is deprived of viral genes, thus the vector as a vaccine carrier is non-immunogenic and may be inoculated for multiple times for re-vaccination. The “gutless” adenovirus vector also contains 36 kb space for accommodating transgenes, thus allowing co-delivery of a large number of antigen genes into cells. Specific sequence motifs such as the RGD motif may be inserted into the H-I loop of an adenovirus vector to enhance its infectivity. An adenovirus recombinant may be constructed by cloning specific transgenes or fragments of transgenes into any of the adenovirus vectors such as those described below. The adenovirus recombinant vector is used to transduce epidermal cells of a vertebrate in a non-invasive mode for use as an immunizing agent. The adenovirus vector may also be used for invasive administration methods, such as intravenous, intramuscular, or subcutaneous injection.

The present Ad-vector GnRH vaccines encode a recombinant protein, which may comprise a carrier immunogenic antigen(s), and mammalian GnRH sequence(s) or homolog(s) thereof, wherein the expressed recombinant protein contains a number of immunogenic sites for inducing antibody generation against host GnRH. In certain embodiments, the anti-GnRH antibodies are neutralizing antibodies. In embodiments, the carrier antigen is a bacterial antigen. In certain embodiments, the Ad-vector GnRH composition comprises one more carrier antigen genes, that may the same or different. In certain embodiments, the Ad-vector GnRH composition comprises LKT, Hep B core antigen and B. anthracis lethal factor. The antigen may be full length, or the vector may encode an immunogenic fragment of the carrier antigen.

GnRH is a 10 amino acid peptide that is conserved across all mammals. In embodiments, GnRH is a natural or endogenous peptide, represented by Glu-His-Trp-Ser-Tyr-Gly-Leu-Arg-Pro-Gly (SEQ ID NO: 2), or any peptide that mimics (also referred to herein as an agonist or antagonist) endogenous GnRH and will induce antibody generation. GnRH mimics include a homolog, analog, ortholog or derivative there. In certain embodiments, the GnRH sequence differs from the endogenous sequence by one amino acid, two amino acids, three amino acids or by four amino acids. The GnRH sequence may also be modified wherein side groups have been added to one or more amino acids to increase the immunogenicity of the GnRH peptide. In certain embodiments, one or more amino acids may be substituted with a non-natural amino acid to increase the immunogenicity of the GnRH peptide or with amino acids lacking the modifications found in naturally processed GnRH within a mammalian host.

In embodiments, the expressed recombinant protein may comprise linear repeats, or a fragment thereof, of GnRH. Those repeats or monomers may be from about 3 to about 20, which may optionally be linked by a short amino acid sequence, 3 to about 6 amino acids, between each repeat sequence. However, the linker may be longer or it may be shorter, wherein the linker is involved in the folding of the expressed recombinant protein. In embodiments, the GnRH repeat sequences may be grouped together or they may be before, or after, the carrier antigen, or both. For example, an Ad-vectored GnRH vaccine may express a recombinant protein wherein a group of GnRH repeat sequences, such as from about 3 to about 10 are present before the carrier antigen followed by a second group of GnRH repeat sequences from about 3 to about 10.

To increase the immunogenicity of the GnRH epitopes, the Ad-vectored GnRH vaccine may comprise heterogeneous carrier antigens. The carrier antigen is immunogenic and facilitates the host generating an immune response against the co-expressed GnRH sequence. Particularly, useful carrier antigens are those immunogenic antigens, or fragments thereof, derived from bacteria or viruses. In other words, the carrier antigen may comprise bacterial or viral antigens, or immunogenic fragments thereof. In embodiments, the carrier antigen does not comprise an endogenous (host) cancer antigen or any self-antigen. In embodiments, the carrier antigen includes a B. anthracis antigen, which may be lethal factor antigen or protective antigen, or an immunogenic fragment thereof. In embodiments, the protective antigen may be PA83, PA63 or an immunogenic fragment thereof. In certain embodiments the expressed recombinant protein may comprise a combination of B. anthracis carrier antigen and/or a Hep B core carrier antigen and/or LKT (leukotoxin antigen, such as from Actinobacillus actinomycetemcomitans or Haemophilus actinomycetemcomitans, Mannheimia haemolytica or Pasteurella haemolytica, Mannheimia glucosida, Bibersteinia trehalosi, Pasteurella multocida, or Staphylococcus aureus). In embodiments, the carrier antigen is selected from leukotoxin antigen, B. anthracis lethal factor, B. anthracis protective antigen, tetanus toxin, Hepatitis B core antigen, keyhole limpet hemocyanin (KLH), bovine serum albumin (BSA), ovalbumin, or a combination thereof.

In certain embodiments, the Ad-vectored GnRH construct may comprise one or more T cell epitopes. In embodiments, the Ad-vectored GnRH construct comprises one or more T cell epitopes between 8 and 11 amino acids in length. In embodiments, the Ad-vectored GnRH construct comprises one or more T cell epitopes between 12 and 25 amino acids in length. In embodiments, the T cell epitopes are selected from those present on bacterial or viral pathogens. Examples include any of those present in influenza antigens, hepatitis B antigens, hepatitis C antigens. In certain embodiments, T cell epitopes used in the present Ad GnRH construct may also be derived from self-cancer antigens. See for example, WO 2009/027688; WO 2014/102540; and, WO 2015/033140.

In exemplary embodiments, an Ad-vectored GnRH vaccine of interest contains and expresses a nucleic acid encoding a recombinant protein, which may comprise: 1) 8 multimers of GnRH positioned before and following the lethal factor reading frame; 2) 8 multimers of GnRH positioned before and following the lethal factor plus Hep B core antigen reading frame; or 3) 8 multimers of GnRH positioned before and following the protective antigen PA83 reading frame. In embodiments, construct may be codon-optimized for cell expression, such as dog cell expression. In certain embodiments, the constructs may comprise linker sequences between the GnRH multimers or between the carrier antigen and the GnRH sequence.

In embodiments, the immunogenic compositions or Ad-vectored GnRH vaccines of interest may be formulated for administration to the mammal. With respect to dosages, routes of administration, formulations, adjuvants, and uses for recombinant viruses and expression products therefrom, compositions of the invention may be used for parenteral or mucosal administration, preferably by intradermal, subcutaneous, intranasal or intramuscular routes. When mucosal administration is used, it is possible to use oral, ocular or nasal routes.

The formulations which may comprise the adenovirus vector of interest, can be prepared in accordance with standard techniques well known to those skilled in the pharmaceutical or veterinary art. Such formulations can be administered in dosages and by techniques well known to those skilled in the veterinary arts taking into consideration such factors as the age, sex, weight, and the route of administration. The formulations can be administered alone, or can be co-administered or sequentially administered with compositions, e.g., with “other” immunological composition, or attenuated, inactivated, recombinant vaccine or therapeutic compositions thereby providing multivalent or “cocktail” or combination compositions of the invention and methods employing them. In embodiments, the formulations comprise sucrose as a cryoprotectant and polysorbate-80 as a non-ionic surfactant. In certain embodiments, the formulations further comprise free-radical oxidation inhibitors ethanol and histidine, the metal-ion chelator ethylenediaminetetraacetic acid (EDTA), or other agents with comparable activity (e.g block or prevent metal-ion catalyzed free-radical oxidation).

The formulations may be present in a liquid preparation for mucosal administration, e.g., oral, nasal, ocular, etc., formulations such as suspensions and, preparations for parenteral, subcutaneous, intradermal, intramuscular, intravenous (e.g., injectable administration) such as sterile suspensions or emulsions. In such formulations the adenoviral vector may be in admixture with a suitable carrier, diluent, or excipient such as sterile water, physiological saline, or the like. The formulations can also be lyophilized or frozen. The formulations can contain auxiliary substances such as wetting or emulsifying agents pH buffering agents, adjuvants, preservatives, and the like, depending upon the route of administration and the preparation desired. The formulations can contain at least one adjuvant compound.

In embodiments, the adjuvant may be GMCSF, or a TLR3 agonist, such as CpG or poly-ICLC; or the adjuvant may be any other adjuvant which enhances the immunogenicity of the recombinant protein and the GnRH epitopes and is compatible with an Ad-vectored vaccine. There is no intended limitation on the choice or use of the adjuvant.

In embodiments, a solution of adjuvant according to the disclosure, is prepared in distilled water, optionally in the presence of sodium chloride, the solution obtained. The stock solution is diluted by adding it to the desired quantity (for obtaining the desired final concentration), or a substantial part thereof, of water charged with NaCl, preferably physiological saline (NaCl 9 g/l) all at once in several portions with concomitant or subsequent neutralization (pH 7.3 to 7.4), preferably with NaOH. This solution at physiological pH will be used as it is for mixing with the vaccine, which may be especially stored in freeze-dried, liquid or frozen form.

Standard texts, such as “REMINGTON'S PHARMACEUTICAL SCIENCE”, 17th edition, 1985, incorporated herein by reference, may be consulted to prepare suitable preparations, without undue experimentation.

Methods of Use

In embodiments, herein are provided methods for inducing an immune response against GnRH in a mammal, which comprises the step of administering the present immunogenic composition (e.g. Ad-GnRH vector construct) to a mammal. In certain embodiments, herein are provided methods for inducing infertility via GnRH autoantibody generation in a mammal, which may comprise the step of: contacting the animal in a non-invasive mode (e.g., skin/mucosal/intranasal area of the animal) or via injection (intramuscular, subcutaneous or intravenous) with an Ad-vector GnRH vaccine and optionally an adjuvant wherein the amount of the vaccine and the optional adjuvant is an amount effective to induce an immune response against host GnRH in the animal. In certain other embodiments, herein are provided methods for inducing infertility via GnRH autoantibody generation in a mammal utilizing a heterologous prime boost dosing regimen, comprising first administering the present immunogenic composition (e.g. Ad-GnRH vector construct) followed by administering a second composition comprising a GnRH-protein construct.

In embodiments, the present methods comprise a homologous prime boost dosing regimen wherein the same Ad-vectored GnRH construct is administered as a prime dose and the boost dose administered from about 4 weeks to about 52 weeks after the prime dose. In certain embodiments, the present methods comprise a heterologous prime boost dosing regimen wherein the present Ad-vectored GnRH construct is administered as a prime dose and a different GnRH composition, such as a GnRH-carrier protein construct, is administered as a boost dose about 4 weeks to about 52 weeks after the prime dose. See Example 5. An example of a GnRH-carrier protein vaccine that can be used as a heterologous boost dose is the GnRH-protein vaccine Equity® Oestrus Control Vaccine for Horses, which is a GnRH peptide conjugated to diphtheria toxoid. In embodiments, the heterologous boost dose is a GnRH carrier protein conjugate selected from GnRH-diphtheria toxoid conjugate, GnRH-tetanus toxin conjugate, GnRH-lethal factor conjugate, GnRH-protective antigen conjugate, GnRH-hepatitis B core antigen conjugate, GnRH-leukotoxin conjugate or a combination thereof.

In embodiments, the prime dose (homologous or heterologous) comprises a present Ad-vector GnRH construct and is administered at day 0. The boost dose may be administered at about week 4 to about week 52 post administration of the prime dose. In certain embodiments, the present Ad-vectored GnRH construct is administered as a boost dose at about week 4 post prime dose administration followed by a heterologous boost dose at about week 48 to about week 52 post administration of the prime dose. In that instance, the prime dose comprises the present Ad-vectored GnRH construct, the first boost dose comprises the present Ad-vectored GnRH construct and the second boost dose comprises a heterologous boost dose of a GnRH-carrier protein conjugate. In embodiments, the heterologous boost dose is administered up to 52 weeks after administration of the prime dose. In certain embodiments, the heterologous boost dose is administered about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks or 52 weeks after administration of the prime dose. In exemplary embodiments, the present Ad-vector GnRH construct was administered as a boost dose at about 4 weeks after administration of the prime dose (comprising the present Ad-vectored GnRH construct) and a second boost dose comprise a heterologous dose of a GnRH-carrier protein was administered at about 49 weeks after administration of the prime dose. See Example 5.

In embodiments, the mammal is a companion animal, a domesticated animal, a feral animal, a food-or feed-producing animal, a livestock animal, a game animal, a racing animal, a performance animal, or a sport animal. In certain embodiments, the mammal is a cow, a horse, a dog, a cat, a goat, a sheep, a deer, a coyote or a pig. In embodiments, the infertility induced by generation of anti-GnRH antibodies lasts for a period of at least about 1 to 12 months, about 2 to 4 years or at least 4 years after the initial administration (prime dose) of the immunogenic composition comprising a present Ad-vectored GnRH construct.

In certain embodiments the dosage of the Ad-vector GnRH vaccine to induce an immune response is lower than compared to an Ad-vectored GnRH vaccine used without an adjuvant. Dosage of the Ad-vector GnRH vaccine when used with or without an adjuvant may range from about 10⁶ to about 10¹² infectious unit or plaque forming unit (ifu or pfu), or the dosage unit may be a viral particle (vp), wherein 1 vp equals about 1-100 ifu or pfu. In one embodiment the dose of Ad-vector GnRH vaccine administered to the animal is about, or at least about, 10⁶ ifu or pfu. In another aspect the dose of Ad-vector GnRH vaccine administered to the animal is about, or at least about, 10⁷ ifu or pfu. In yet another aspect, the dose of Ad-vector GnRH vaccine administered to the animal is about, or at least about, 10⁸ ifu or pfu. In another aspect the dose of Ad-vector GnRH vaccine administered to the animal is about, or at least about, 10⁹ ifu or pfu. In another aspect the dose of Ad-vector GnRH vaccine administered to the animal is about, or at least about, 10¹⁰ ifu or pfu. In yet another aspect, the dose of Ad-vector GnRH vaccine administered to the animal is about, or at least about, 10¹¹ ifu or pfu. In another aspect the dose of Ad-vector GnRH vaccine administered to the animal is about, or at least about, 10¹² ifu or pfu.

One of skill in the art understands that an effective dose in a mouse may be scaled for larger animals such as dogs, horses, pigs, etc. In that way, through allometric scaling (also referred to as biological scaling) a dose in a larger animal may be extrapolated from a dose in a mouse to obtain an equivalent dose based on body weight or body surface area of the animal.

In certain embodiments, non-invasive administration of the Ad-vector GnRH vaccine includes, but is not limited to, topical application to the skin, and/or intranasal and/or mucosal and/or perlingual and/or buccal and/or oral and/or oral cavity administration. Dosage forms for the application of the Ad-vector GnRH vaccine may include liquids, ointments, powders and sprays. The active component may be admixed under sterile conditions with a physiologically acceptable carrier and any preservative, buffers, propellants, or absorption enhancers as may be needed.

If nasal or respiratory (mucosal) administration is desired, compositions may be in a form and dispensed by a squeeze spray dispenser, pump dispenser, multi-dose dispenser, dropper-type dispenser or aerosol dispenser. Such dispensers may also be employed to deliver the composition to oral or oral cavity (e.g., buccal or perlingual) mucosa. Aerosols are usually under pressure by means of a hydrocarbon. Pump dispensers may preferably dispense a metered dose or, a dose having a particular particle size.

While non-invasive delivery may be desirable under some circumstances for administration of the Ad-vectored GnRH vaccine administration by injection may also be used to administer the Ad-vectored GnRH vaccine, such as via intramuscular, subcutaneous or intravenous injection.

An immunological effective amount, as used herein refers to an amount or concentration of the Ad-vector GnRH vaccine encoding and expressing the recombinant protein of interest, that when administered to a subject, produces an immune response to the host GnRH. The Ad-vector GnRH vaccines of the present disclosure may be administered to an animal either alone or as part of an immunological composition.

The immunogenic compositions may contain pharmaceutically acceptable flavors and/or colors for rendering them more appealing, especially if they are administered orally (or buccally or perlingually); and, such compositions may be in the form of tablets or capsules that dissolve in the mouth or which are bitten to release a liquid for absorption buccally or perlingually (akin to oral, perlingual or buccal medicaments). The viscous compositions may be in the form of gels, lotions, ointments, creams and the like (e.g., for topical and/or mucosal and/or nasal and/or oral and/or oral cavity and/or perlingual and/or buccal administration), and will typically contain a sufficient amount of a thickening agent so that the viscosity is from about 2500 to 6500 cps, although more viscous compositions, even up to 10,000 cps may be employed.

Liquid preparations are normally easier to prepare than gels, other viscous compositions, and solid compositions. Additionally, liquid compositions are somewhat more convenient to administer, especially by orally or buccally or perlinually, to animals, in single or in multi-dose preparations. Viscous compositions, on the other hand, may be formulated within the appropriate viscosity range to provide longer contact periods with mucosa, such as the lining of the stomach or nasal mucosa or for perlingual or buccal or oral cavity absorption.

The Ad-vector may be matched to the host or may be a vector that is interesting to employ with respect to the host or animal because the vector may express both heterologous or exogenous and homologous gene products of interest in the animal; for instance, in veterinary applications, it may be useful to use a vector pertinent to the animal, for example, in canines one may use canine adenovirus; or more generally, the vector may be an attenuated or inactivated natural pathogen of the host or animal upon which the method is being performed. One skilled in the art, with the information in this disclosure and the knowledge in the art, may match a vector to a host or animal without undue experimentation.

Therefore, the method of the disclosure may be used to immunize a companion animal, a domesticated animal, a feral animal, a food-or feed-producing animal, a livestock animal, a game animal, a racing animal, a performance animal, or a sport animal. The term animal means all mammals. Examples of mammals include horses, cows, dogs, cats, goats, sheep, birds and pigs, etc. Since the immune systems of all vertebrates operate similarly, the applications described may be implemented in all mammalian vertebrate systems.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

The Examples below are given so as to illustrate the practice of this invention. They are not intended to limit or define the entire scope of this invention.

### Example 1: Construction of GnRH Recombinant Adenovirus Vectors

Components of the antigen cassette include: sixteen multimers of native GnRH sequence linked head to tail, arranged so the at least one GnRH repeat has a free C-terminus which is required to mimic the active hormone for recognition by GnRH receptors. The N-terminus of GnRH (n6) will be amide linked with a 7 base hinge region of IgG (amino acid residues 225-232) to allow conformational changes mimicking the native hormone. The C terminus of IgG Hinge is linked to an antigenic carrier. Carriers include lethal factor of Bacillus anthracis, lethal factor plus the core antigen of human hepatitis B, protective antigen (P83) of Bacillus anthracis, and the leukotoxin fragment of Pasteurella haemolytica. All nucleotides will be codon optimized for the target species.

GnRH multimer-Lethal Factor (LFn) of Bacillus anthracis Antigen Cassette

LFn fragment (aa 1-254) is a truncated version of lethal factor which retained the N terminal PA-binding domain. The GnRH antigen cassette used in constructing the AdGnRHLFn antigen consists of 8 multimers of GnRH positioned before and following the LFn reading frame. The sequence is preceded by a HindIII ribosomal binding site followed by an ATG and TPA expression sequence shown bold below. This is followed by the GnRH multimers, with each GnRH decapeptide separated by a spacer shown in underline below. A hinge fragment 225-232/225′-232′ of human IgG1(bold) and a T helper peptide from canine distemper virus protein and other T-helper sequences (italics). The LFn fragment is followed by a 9-nucleic acid sequence. The sequences following the LFn fragment consist of T helper, Hinge and GnRH multimer. These sequences are identical to those preceding the LFn carrier. The cassette is terminated with a XbaI-TAGTAGTCTAGA (SEQ ID NO: 3) stop codon. This construction is typical of all antigen cassettes prepared and differs only in the carrier epitope.

GnRH8-Hinge-Thelp-LFn-Thelp-hinge-GnRH8
Nucleic Acid Sequence
(SEQ ID NO: 4)
AAGCTTACCATGGATGCAATGAAGAGAGGGCTCTGCTGTGTGCTGCTGCTGTGTGG
AGCAGTCTTCGTTTCGCCCAGCGGTACCGGATCCGAACACTGGTCTTACGGGCTGAGGCCTG
GCAGTGGATCCGAGCATTGGTCTTACGGCCTGAGACCTGGCGGCAGTTCTGAACACTGGTCA
TACGGACTCAGACCAGGAAGCGGCTCTGAGCATTGGTCTTATGGCCTGCGCCCTGGCGGCAG
TTCTGAACACTGGTCATACGGACTCAGACCAGGAAGCGGCTCTGAGCATTGGTCTTATGGCC
TGCGCCCTGGCGGCAGTTCTGAACATTGGTCTTACGGACTGAGGCCTGGAAGTGGATCCGAG
CACTGGTCTTATGGCCTCCGCCCTGGCGGCAGTTCTACTTGTCCTCCCTGCCCAGCTCCGA
GCGGCTCTACTGCTGCCCAAATTACCGCAGGTATCGCGCTTCATCAGTCTAATCTCAACGGCAGTT
CTAAACTTATTCCTAATGCTTCTCTCATCGAAAACTGTACTAAGGCGGAGTTGAGCGGCTCTGCGGA
GTTGGGGGAGTACGAAAAGCTACTCAACTCGGTGCTTGAACCCATCGGCAGTTCTGAATATCTCAA
CAAAATCCAAAATAGCCTAAGTACAGAGTGGAGTCCTTGTAGCGTAACGAGCGGCTCTGTCAGTCC
CTTCTATAGCTACATCAAAACAAGTAATATTCTGGCAGATGACGTAGGCAGTTCTGCTAAAGCCGTA
GCAGCGTGGACACTAAAGGCTGCCGCAAGCGGCTCCGCCGGCGGCCACGGCGATGTGGGCAT
GCACGTGAAAGAGAAAGAGAAAAACAAAGATGAGAACAAGCGGAAAGATGAAGAGCGGA
ACAAAACCCAGGAAGAGCACCTGAAGGAAATCATGAAACACATCGTGAAAATCGAAGTGA
AAGGCGAGGAAGCTGTGAAAAAGGAGGCCGCTGAAAAGCTGCTCGAGAAAGTGCCCAGCG
ATGTGCTGGAGATGTACAAAGCCATCGGCGGCAAAATCTACATCGTGGATGGCGATATCAC
CAAACACATCAGCCTGGAAGCCCTGAGCGAAGATAAGAAAAAGATCAAAGACATCTACGGC
AAAGATGCTCTGCTCCACGAACACTACGTGTACGCCAAAGAAGGCTACGAACCCGTGCTCGT
GATCCAGAGCAGCGAAGATTACGTGGAAAACACCGAAAAGGCCCTGAACGTGTACTACGAA
ATCGGCAAGATCCTGAGCCGGGATATCCTGAGCAAAATCAACCAGCCCTACCAGAAATTCCT
GGATGTGCTGAACACCATCAAAAACGCCAGCGATAGCGATGGCCAGGATCTGCTCTTCACC
AACCAGCTGAAGGAACACCCCACCGACTTCAGCGTGGAATTCCTGGAACAGAACAGCAACG
AGGTGCAGGAAGTGTTCGCCAAAGCTTTCGCCTACTACATCGAGCCCCAGCACCGGGATGTG
CTGCAGCTGTACGCCCCCGAAGCTTTCAACTACATGGATAAATTCAACGAACAGGAAATCAA
CCTGGCCGGCAAGACTGCTGCCCAAATTACCGCAGGTATCGCGCTTCATCAGTCTAATCTCAACG
GCAGTTCTAAACTTATTCCTAATGCTTCTCTCATCGAAAACTGTACTAAGGCGGAGTTGAGCGGCTC
TGCGGAGTTGGGGGAGTACGAAAAGCTACTCAACTCGGTGCTTGAACCCATCGGCAGTTCTGAAT
ATCTCAACAAAATCCAAAATAGCCTAAGTACAGAGTGGAGTCCTTGTAGCGTAACGAGCGGCTCTG
TCAGTCCCTTCTATAGCTACATCAAAACAAGTAATATTCTGGCAGATGACGTAGGCAGTTCTGCTAA
AGCCGTAGCAGCGTGGACACTAAAGGCTGCCGCAAGCGGCTCCACTTGTCCTCCCTGCCCAG
CTCCGAGCGGCTCTGAACACTGGTCTTACGGGCTGAGGCCTGGCAGTGGATCCGAGCATTG
GTCTTACGGCCTGAGACCTGGCGGCAGTTCTGAACACTGGTCATACGGACTCAGACCAGGAA
GCGGCTCTGAGCATTGGTCTTATGGCCTGCGCCCTGGCGGCAGTTCTGAACACTGGTCATAC
GGACTCAGACCAGGAAGCGGCTCTGAGCATTGGTCTTATGGCCTGCGCCCTGGCGGCAGTTC
TGAACATTGGTCTTACGGACTGAGGCCTGGAAGTGGATCCGAGCACTGGTCTTATGGCCTCC
GCCCTGGCGGCAGTTCTTAGTAGTCTAGA
Amino Acid Sequence
Start-TPA
(SEQ ID NO: 5)
MDAMKRGLCCVLLLCGAVFVSPSGTGS
8 copies GnRH + spacers
(SEQ ID NO: 6)
EHWSYGLRPGSGS EHWSYGLRPGGSS EHWSYGLRPGSGS EHWSYGLRPGGSS
EHWSYGLRPGSGS EHWSYGLRPGGSS EHWSYGLRPGSGS EHWSYGLRPG
Hinge
(SEQ ID NO: 7)
TCPPCPAP
CDV T-help
(SEQ ID NO: 8)
KLIPNASLIENCTKAEL
Influenza Hemaglutinin T-help
(SEQ ID NO: 9)
GALNNRFQIKGVELKS
Start LFn
(SEQ ID NO: 10)
QEEHLKEIMK HIVKIEVKGE EAVKKEAAEK LLEKVPSDVL EMYKAIGGKI
YIVDGDITKH ISLEALSEDK KKIKDIYGKD ALLHEHYVYA KEGYEPVLVI QSSEDYVENT
EKALNVYYEI GKILSRDILS KINQPYQKFLD VLNTIKNASD SDGQDLLFTNQLKEHPTDFS
VEFLEQNSNE VQEVFAKAFA YYIEPQHRDVLQLYAPEAFN YMDKFNEQEI NL End LFn
CDV T-help
(SEQ ID NO: 8)
KLIPNASLIENCTKAEL
Influenza Hemaglutinin T-help
(SEQ ID NO: 9)
GALNNRFQIKGVELKS
Hinge
(SEQ ID NO: 7)
TCPPCPAP
8 copies GnRH + spacers
(SEQ ID NO: 6)
EHWSYGLRPGSGS EHWSYGLRPGGSS EHWSYGLRPGSGSEHWSYGLRP GGSS
EHWSYGLRPGSGS EHWSYGLRPGGSS EHWSYGLRPGSGS EHWSYGLRPG

GnRH multinier-Lethal Factor Plus Hepatitis B Core Antigen Cassette

This antigen consists of all of the same sequences described in the GnRH multimer-Lethal Factor Antigen Cassette with the addition of sequences for the antigenic component of human hepatitis B core antigen.

GnRH8-Hinge-Thelp-LFn-HBc-Thelp-hinge-GnRH8
Nucleic Acid Sequence
(SEQ ID NO: 11)
AAGCTTACCATGGATGCAATGAAGAGAGGGCTCTGCTGTGTGCTGCTGCTGTGTGG
AGCAGTCTTCGTTTCGCCCAGCGGTACCGGATCCGAACACTGGTCTTACGGGCTGAGGCCTG
GCAGTGGATCCGAGCATTGGTCTTACGGCCTGAGACCTGGCGGCAGTTCTGAACACTGGTCA
TACGGACTCAGACCAGGAAGCGGCTCTGAGCATTGGTCTTATGGCCTGCGCCCTGGCGGCAG
TTCTGAACACTGGTCATACGGACTCAGACCAGGAAGCGGCTCTGAGCATTGGTCTTATGGCC
TGCGCCCTGGCGGCAGTTCTGAACATTGGTCTTACGGACTGAGGCCTGGAAGTGGATCCGAG
CACTGGTCTTATGGCCTCCGCCCTGGCGGCAGTTCTACTTGTCCTCCCTGCCCAGCTCCGAGC
GGCTCTACTGCTGCCCAAATTACCGCAGGTATCGCGCTTCATCAGTCTAATCTCAACGGCAG
TTCTAAACTTATTCCTAATGCTTCTCTCATCGAAAACTGTACTAAGGCGGAGTTGAGCGGCTC
TGCGGAGTTGGGGGAGTACGAAAAGCTACTCAACTCGGTGCTTGAACCCATCGGCAGTTCTG
AATATCTCAACAAAATCCAAAATAGCCTAAGTACAGAGTGGAGTCCTTGTAGCGTAACGAG
CGGCTCTGTCAGTCCCTTCTATAGCTACATCAAAACAAGTAATATTCTGGCAGATGACGTAG
GCAGTTCTGCTAAAGCCGTAGCAGCGTGGACACTAAAGGCTGCCGCAAGCGGCTCCGCCGG
CGGCCACGGCGATGTGGGCATGCACGTGAAAGAGAAAGAGAAAAACAAAGATGAGAACAA
GCGGAAAGATGAAGAGCGGAACAAAACCCAGGAAGAGCACCTGAAGGAAATCATGAAACA
CATCGTGAAAATCGAAGTGAAAGGCGAGGAAGCTGTGAAAAAGGAGGCCGCTGAAAAGCT
GCTCGAGAAAGTGCCCAGCGATGTGCTGGAGATGTACAAAGCCATCGGCGGCAAAATCTAC
ATCGTGGATGGCGATATCACCAAACACATCAGCCTGGAAGCCCTGAGCGAAGATAAGAAAA
AGATCAAAGACATCTACGGCAAAGATGCTCTGCTCCACGAACACTACGTGTACGCCAAAGA
AGGCTACGAACCCGTGCTCGTGATCCAGAGCAGCGAAGATTACGTGGAAAACACCGAAAAG
GCCCTGAACGTGTACTACGAAATCGGCAAGATCCTGAGCCGGGATATCCTGAGCAAAATCA
ACCAGCCCTACCAGAAATTCCTGGATGTGCTGAACACCATCAAAAACGCCAGCGATAGCGA
TGGCCAGGATCTGCTCTTCACCAACCAGCTGAAGGAACACCCCACCGACTTCAGCGTGGAAT
TCCTGGAACAGAACAGCAACGAGGTGCAGGAAGTGTTCGCCAAAGCTTTCGCCTACTACATC
GAGCCCCAGCACCGGGATGTGCTGCAGCTGTACGCCCCCGAAGCTTTCAACTACATGGATAA
ATTCAACGAACAGGAAATCAACCTGGCCGGCATGATCGACATTGACACGTATAAAGAATTT
GGAGCTTCTGTGGAGTTACTCTCTTTTTTGCCTTCTGACTTCTTTCCTTCTATTCGTGATCTCC
TCGACACCGCCTCTGCTCTGTATCGGGAGGCCTTAGAGTCTCCGGAACATTGTTCACCTCACC
ATACAGCACTAAGGCAAGCTATTCTGTGTTGGGGTGAGTTGATGAATCTGGCCACCTGGGTG
GGAAGTAATTTGGAAGACCCAGCATCCAGGGAATTAGTAGTAAGCTATGTCAATGTTAATAT
GGGCCTAAAAATCAGACAACTATTATGGTTTCACATTTCCTGTCTTACTTTTGGAAGAGAAA
CTGTTCTTGAGTATTTGGTGTCTTTTGGAGTGTGGATTCGCACTCCTCCCGCTTACAGACCAC
CAAATGCCCCTATCTTATCAACACTTCCGGAAACTACTGTTGTTGCCGGCATGACTGCTGCCC
AAATTACCGCAGGTATCGCGCTTCATCAGTCTAATCTCAACGGCAGTTCTAAACTTATTCCTA
ATGCTTCTCTCATCGAAAACTGTACTAAGGCGGAGTTGAGCGGCTCTGCGGAGTTGGGGGAG
TACGAAAAGCTACTCAACTCGGTGCTTGAACCCATCGGCAGTTCTGAATATCTCAACAAAAT
CCAAAATAGCCTAAGTACAGAGTGGAGTCCTTGTAGCGTAACGAGCGGCTCTGTCAGTCCCT
TCTATAGCTACATCAAAACAAGTAATATTCTGGCAGATGACGTAGGCAGTTCTGCTAAAGCC
GTAGCAGCGTGGACACTAAAGGCTGCCGCAAGCGGCTCCACTTGTCCTCCCTGCCCAGCTCC
GAGCGGCTCTGAACACTGGTCTTACGGGCTGAGGCCTGGCAGTGGATCCGAGCATTGGTCTT
ACGGCCTGAGACCTGGCGGCAGTTCTGAACACTGGTCATACGGACTCAGACCAGGAAGCGG
CTCTGAGCATTGGTCTTATGGCCTGCGCCCTGGCGGCAGTTCTGAACACTGGTCATACGGAC
TCAGACCAGGAAGCGGCTCTGAGCATTGGTCTTATGGCCTGCGCCCTGGCGGCAGTTCTGAA
CATTGGTCTTACGGACTGAGGCCTGGAAGTGGATCCGAGCACTGGTCTTATGGCCTCCGCCC
TGGCGGCAGTTCTTAGTAGTCTAGA
Amino Acid Sequence
(SEQ ID NO: 12)
MDAMKRGLCCVLLLCGAVFVSPSGTGSEHWSYGLRPGSGSEHWSYGLRPGGSSEHWS
YGLRPGSGSEHWSYGLRPGGSSEHWSYGLRPGSGSEHWSYGLRPGGSSEHWSYGLRPGSGSEH
WSYGLRPGTCPPCPAPKLIPNASLIENCTKAELGALNNRFQIKGVELKSQEEHLKEIMKHIVKIEV
KGEEAVKKEAAEKLLEKVPSDVLEMYKAIGGKIYIVDGDITKHISLEALSEDKKKIKDIYGKDAL
LHEHYVYAKEGYEPVLVIQSSEDYVENTEKALNVYYEIGKILSRDILSKINQPYQKFLDVLNTIKN
ASDSDGQDLLFTNQLKEHPTDFSVEFLEQNSNEVQEVFAKAFAYYIEPQHRDVLQLYAPEAFNY
MDKFNEQEINLIDIDTYKEFGASVELLSFLPSDFFPSIRDLLDTASALYREALESPEHCSPHHTALR
QAILCWGELMNLATWVGSNLEDPASRELVVSYVNVNMGLKIRQLLWFHISCLTFGRETVLEYLV
SFGVWIRTPPAYRPPNAPILSTLPETTVVKLIPNASLIENCTKAELGALNNRFQIKGVELKSTCPPCP
APEHWSYGLRPGSGSEHWSYGLRPGGSSEHWSYGLRPGSGSEHWSYGLRPGGSSEHWSYGLRP
GSGSEHWSYGLRPGGSSEHWSYGLRPGSGSEHWSYGLRPG

GnRH multimer-PA83 Antigen Cassette

PA83 sequence is derived from the humanized PA gene which was used for generation AdtPA83hu.

Nucleic Acid Sequence
(SEQ ID NO: 13)
AAGCTTCCACCAAACTCATCCCAAACGCATCACTCATCGAGAACTGCACCAAGGCTGAACTG
GAGGTCAAACAGGAGAATAGGCTGCTGAACGAGAGCGAGAGCTCCTCTCAGGGCCTGCTCG
GATACTATTTTTCCGACCTGAACTTCCAGGCTCCAATGGTGGTCACAAGTTCAACCACAGGG
GATCTCTCAATCCCCAGCTCCGAGCTGGAAAATATTCCTAGCGAGAACCAGTACTTTCAGTC
TGCCATCTGGAGTGGCTTCATTAAGGTGAAGAAATCTGACGAGTATACCTTTGCTACAAGTG
CAGATAATCACGTGACCATGTGGGTCGACGATCAGGAAGTGATCAACAAGGCCTCCAACTC
TAATAAGATTCGCCTCGAGAAAGGTCGACTGTACCAGATCAAAATTCAGTATCAGCGGGAG
AACCCTACCGAAAAGGGCCTCGACTTCAAACTGTACTGGACAGATTCTCAGAATAAGAAAG
AAGTGATCTCTAGTGACAACCTCCAGCTGCCAGAGCTGAAGCAGAAATCAAGCAATTCCCG
AAAGAAAAGGAGTACATCAGCCGGCCCCACTGTGCCTGACAGGGATAACGACGGAATCCCA
GACTCTCTGGAGGTCGAAGGGTATACTGTGGATGTCAAGAACAAGAGAACCTTTCTGTCACC
CTGGATCAGCAACATTCATGAAAAGAAAGGACTGACTAAGTACAAATCCTCTCCAGAGAAG
TGGAGTACCGCCTCAGATCCCTATAGCGACTTCGAAAAGGTGACAGGGCGGATCGACAAAA
ACGTCAGCCCTGAGGCTCGACACCCACTGGTGGCAGCTTACCCCATTGTGCATGTCGATATG
GAGAATATCATTCTGTCCAAGAACGAAGACCAGTCTACTCAGAATACCGATAGTGAGACTC
GAACCATCTCAAAGAACACAAGCACTTCCAGGACCCACACATCCGAGGTGCATGGAAACGC
TGAAGTCCACGCATCTTTCTTTGACATCGGCGGATCTGTGAGTGCTGGGTTCTCAAACAGCA
ATAGTTCAACCGTCGCAATTGATCATTCCCTCTCTCTGGCCGGCGAGAGGACATGGGCTGAA
ACTATGGGCCTCAACACAGCCGACACTGCTAGGCTGAACGCAAATATCAGATACGTGAATA
CTGGAACCGCCCCAATCTACAACGTGCTGCCCACTACCTCCCTCGTCCTGGGGAAGAATCAG
ACCCTGGCCACAATCAAGGCTAAAGAGAACCAGCTCAGTCAGATTCTGGCCCCCAACAATT
ACTATCCTTCTAAGAATCTCGCACCTATCGCCCTGAACGCTCAGGACGATTTTAGCTCCACTC
CAATTACCATGAACTACAATCAGTTCCTCGAGCTGGAAAAGACCAAACAGCTCCGGCTGGAT
ACAGACCAGGTGTACGGAAATATCGCAACATATAACTTTGAGAATGGTCGGGTGCGCGTCG
ACACTGGCAGCAACTGGTCCGAGGTGCTGCCTCAGATCCAGGAAACAACTGCCCGCATCATT
TTCAATGGCAAGGATCTCAACCTGGTGGAGAGGAGAATCGCAGCCGTCAACCCAAGCGACC
CCCTGGAGACCACAAAGCCCGATATGACCCTCAAGGAAGCACTGAAAATCGCCTTCGGGTTT
AATGAGCCTAACGGTAATCTGCAGTACCAGGGCAAGGACATCACAGAATTCGATTTTAACTT
CGACCAGCAGACTAGCCAGAACATTAAGAATCAGCTCGCTGAGCTGAACGCAACCAATATC
TACACAGTGCTCGACAAGATCAAGCTGAACGCTAAGATGAATATCCTGATTAGAGATAAAC
GGTTCCACTATGACCGCAACAATATCGCAGTGGGTGCCGATGAATCCGTGGTCAAGGAGGC
ACATCGAGAAGTCATCAATTCTAGTACCGAGGGACTGCTCCTGAACATTGATAAGGACATCA
GGAAAATTCTGTCTGGATACATCGTGGAGATTGAAGACACAGAGGGGCTGAAGGAAGTCAT
CAATGATAGATATGACATGCTCAACATTTCAAGCCTGCGGCAGGATGGCAAGACCTTTATCG
ACTTCAAGAAGTACAACGATAAACTCCCTCTGTACATCAGCAACCCAAATTACAAGGTGAAC
GTCTATGCTGTGACTAAAGAGAACACCATCATTAATCCTAGCGAAAACGGAGACACATCCA
CTAACGGGATCAAGAAAATCCTGATTTTTAGCAAGAAAGGTTACGAAATCGGGGGTGCCCT
CAACAATAGATTCCAGATTAAGGGCGTGGAGCTGAAAAGCTGTAGTCTAGA
Amino Acid Sequence
(SEQ ID NO: 14)
MDAMKRGLCCVLLLCGAVFVSPSGTGSEHWSYGLRPGSGSEHWSYGLRPGGSSEHWS
YGLRPGSGSEHWSYGLRPGGSSEHWSYGLRPGSGSEHWSYGLRPGGSSEHWSYGLRPGSGSEH
WSYGLRPGTCPPCPAPKLIPNASLIENCTKAELGALNNRFQIKGVELKSEVKQENRLLNESESSSQ
GLLGYYFSDLNFQAPMVVTSSTTGDLSIPSSELENIPSENQYFQSAIWSGFIKVKKSDEYTFATSAD
NHVTMWVDDQEVINKASNSNKIRLEKGRLYQIKIQYQRENPTEKGLDFKLYWTDSQNKKEVISS
DNLQLPELKQKSSNSRKKRSTSAGPTVPDRDNDGIPDSLEVEGYTVDVKNKRTFLSPWISNIHEK
KGLTKYKSSPEKWSTASDPYSDFEKVTGRIDKNVSPEARHPLVAAYPIVHVDMENIILSKNEDQS
TQNTDSRTISKNTSTSRTHTSEVHGNAEVHASFFDIGGSVSAGFSNSNSSTVAIDHSLSLAGERTW
AETMGLNTADTARLNANIRYVNTGTAPIYNVLPTTSLVLGKNQTLATIKAKENQLSQILAPNNYY
PSKNLAPIALNAQDDFSSTPITMNYNQFLELEKTKQLRLDTDQVYGNIATYNFENGRVRVDTGSN
WSEVLPQIQETTARIIFNGKDLNLVERRIAAVNPSDPLETTKPDMTLKEALKIAFGFNEPNGNLQY
QGKDITEFDFNFDQQTSQNIKNQLAELNATNIYTVLDKIKLNAKMNILIRDKRFHYDRNNIAVGA
DESVVKEAHREVINSSTEGLLLNIDKDIRKILSGYIVEIEDTEGLKEVINDRYDMLNISSLRQDGKT
FIDFKKYNDKLPLYISNPNYKVNVYAVTKENTIINPSENGDTSTNGIKKILIFSKKGYEIGKLIPNAS
LIENCTKAELGALNNRFQIKGVELKSTCPPCPAPHWSYGLRPGSGSEHWSYGLRPGGSSEHWSY
GLRPGSGSEHWSYGLRPGGSSEHWSYGLRPGSGSEHWSYGLRPGGSSEHWSYGLRPGSGSEHW
SYGLRPG
GnRH multimer-leukotoxin Antigen Cassette
Nucleic Acid Sequence
(SEQ ID NO: 15)
ATGGCAACCGTGATTGACCTCTCATTCCCAAAAACAGGGGCAAAAAAGATTATCCTCTACAT
CCCACAGAACTACCAGTACGACACTGAGCAGGGCAACGGACTGCAGGACCTCGTGAAGGCC
GCTGAGGAACTGGGGATCGAAGTCCAGAGGGAGGAAAGAAACAATATTGCAACTGCCCAG
ACCAGTCTGGGCACAATCCAGACTGCAATTGGCCTGACTGAGCGAGGAATCGTGCTCTCTGC
TCCCCAGATTGATAAGCTGCTCCAGAAGACCAAAGCAGGACAGGCTCTGGGAAGCGCAGAG
TCCATCGTGCAGAACGCAAATAAGGCCAAAACCGTCCTGTCCGGGATCCAGTCTATTCTGGG
GAGTGTGCTCGCCGGTATGGACCTGGATGAGGCCCTCCAGAACAATAGCAACCAGCACGCT
CTGGCAAAAGCCGGACTGGAGCTCACTAATTCTCTGATCGAAAACATTGCCAATAGTGTGAA
GACCCTGGACGAGTTCGGGGAACAGATCTCACAGTTTGGTAGCAAACTGCAGAACATTAAG
GGGCTGGGTACCCTCGGCGATAAGCTGAAAAATATCGGAGGACTCGACAAGGCAGGACTGG
GACTCGATGTGATCTCCGGACTGCTCTCTGGTGCTACCGCAGCACTGGTGCTCGCAGACAAG
AACGCTTCCACAGCAAAGAAAGTCGGGGCAGGTTTCGAGCTGGCCAACCAGGTGGTCGGCA
ATATTACAAAGGCCGTGAGCTCCTATATCCTGGCTCAGAGAGTCGCTGCAGGCCTCTCTAGT
ACAGGACCCGTGGCCGCTCTGATTGCTTCTACTGTCAGTCTGGCAATCTCTCCTCTCGCTTTC
GCAGGAATTGCCGATAAGTTCAACCACGCTAAGTCACTGGAGAGCTACGCCGAACGGTTCA
AGAAACTGGGGTATGACGGCGACAATCTGCTCGCTGAGTACCAGCGCGGCACCGGAACAAT
CGACGCATCCGTGACCGCCATTAACACAGCTCTGGCAGCAATCGCAGGAGGTGTCTCTGCTG
CAGCAGCTAATCTGAAGGATCTCACATTTGAGAAGGTGAAACATAACCTGGTCATCACTAAT
AGCAAGAAAGAAAAAGTGACCATTCAGAACTGGTTCGGCGAGGCCGACTTTGCTAAGGAAG
TGCCAAATTATAAGGCCACTAAAGATGAGAAGATCGAGGAAATCATTGGACAGAACGGGGA
AGGTATCACCAGCAAACAGGTGGACGATCTGATTGCTAAAGGCAACGGAAAGATCACACAG
GACGAGCTGAGCAAGGTGGTCGATAATTACGAACTGCTCAAACATAGCAAGAACGTGACAA
ATTCCCTGGACAAGCTCATCTCAAGCGTCTCAGCCTTCACATCCTCTAACGACAGCGGAAAT
GTGCTGGTCGCTCCCACTAGTATGCTCGATCAGTCACTGAGTTACTCCAGTTTGCCAGGGGG
TCC TAGTAGTCTAGA
Amino Acid Sequence
(SEQ ID NO: 16)
MDAMKRGLCCVLLLCGAVFVSPSGTGSEHWSYGLRPGSGSEHWSYGLRPGGSSEHWS
YGLRPGSGSEHWSYGLRPGGSSEHWSYGLRPGSGSEHWSYGLRPGGSSEHWSYGLRPGSGSEH
WSYGLRPGTCPPCPAPMATVIDLSFPKTGAKKIILYIPQNYQNDTEQGNGLQDLVKAAEELGIEV
QREERNNIATAQTSLGTIQTAIGLTERGIVLSAPQIDKLLQKTKAGQALGSAESIVQNANKAKTVL
SGIQSILGSVLAGMDLDEALQNNSNQHALAKAGLELTNSLIENIANSVKTLDEFGEQISQFGSKLQ
NIKGLGTLGDKLKNIGGLDKAGLGLDVISGLLSGATAALVLADKNASTAKKVGAGFELANQVV
GNITKAVSSYILAQRVAAGLSSTGPVAALIASTVSLAISPLAFAGIADKFNHAKSLESYAERFKKL
GYDGDNLLAEYQRGTGTIDASVTAINTALAAIAGGVSAAAANLKDLTFEKVKHNLVITNSKKEK
VTIQNWFGEADFAKEVPNYKATKDEKIEEIIGQNGEGITSKQVDDLIAKGNGKITQDELSKVVDN
YELLKHSKNVTNSLDKLISSVSAFTSSNDSGNVLVAPTSMLDQSLSSLQFARGSTCPPCPAPEHWS
YGLRPGSGSEHWSYGLRPGGSSEHWSYGLRPGSGSEHWSYGLRPGGSSEHWSYGLRPGSGSEH
WSYGLRPGGSSEHWSYGLRPGSGSEHWSYGLRPG

### Example 2: Mouse Response to GnRH Recombinant Adenovirus Vectors

The study was designed with the single, specific goal of screening candidate adenoviral vectored vaccines in mice to select the most promising one or two vaccine constructs that would then progress to test contraceptive efficacy trials in target species. The study demonstrates Anti-GnRH antibody titers, testosterone values, testicular volumes, and histopathology for five AdGnRH vaccine constructs listed below. Note that Ad-vectored GnRH vaccine compositions 8 and 9 differ only in codon optimization and have the same Leukotoxin antigen.

Ad-vectored vaccine #8: Ad-Leukotoxin-GnRH16M, Mouse codon optimized;

Ad-vectored vaccine #9: Ad-Leukotoxin-GnRH16D, Dog codon optimized;

Ad-vectored vaccine #10: Ad-PA83-GnRH16M, Mouse codon optimized;

Ad-vectored vaccine #11: Ad-LFn-GnRH16M, Mouse codon optimized;

Ad-vectored vaccine #12: Ad-LFn-HBc-GnRH16M, Mouse codon optimized

Study Design

The study protocol consisted of a cross-sectional sampling of mice immunized with a prime dose at approximately 30 days of age, including mice immunized with the Ad-vectored GnRH vaccine alone, mice immunized with the Ad-vectored GnRH vaccine plus the oligonucleotide adjuvant CpG (specific for mice) or adenoviral vectored adjuvant GMCSF (specific for mice) and sampled along with age matched control groups of 3 mice per group at 30, 60, 90 and 120 days after immunization. A booster dose of the same Ad-vectored GnRH vaccine was given at 30, 60 and 90 days.

FIG. 2 shows a fold increase in geometric mean GnRH antibody titers in immunized mice compared to unimmunized control mice.

Those results demonstrate that three of the five vaccines stimulate substantial antibody production by 30 days after primary dosing. Antibody titers increase with time and are sustained through the 120 day duration of the experiments. AdLKTGnRH16 (Ad-vectored GnRH vaccine #8 and #9) induced the earliest, highest and sustained antibody titers. AdLFn-HBcGnRH16 induced antibodies by 30 days and achieved peak titers comparable to the LKT antigen. AdLFnGnRH16 also induced antibodies by 30 days and achieved peak titers comparable to the Ad-vectored GnRH vaccine #8 and #9.

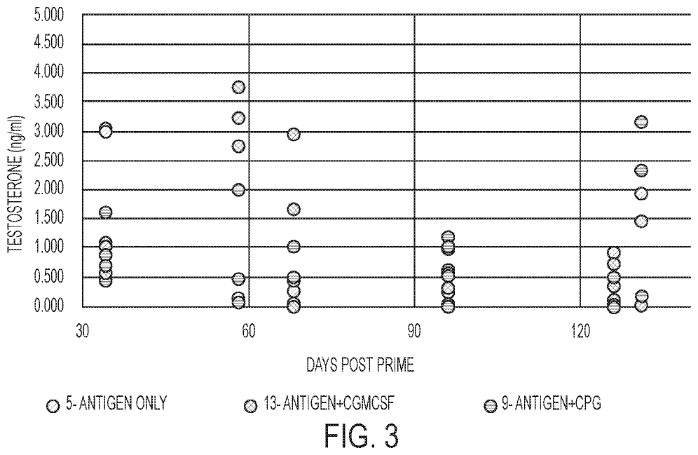

FIG. 3 shows testosterone values for immunized mice of combined Ad-vectored GnRH vaccine #8 and #9 which express the same antigen, AdLKTGnRH16. The data suggests that values below 1 ng/ml may be a threshold for fertility. Data points at or below that threshold are observed at 30 days after a prime immunization and increase with time.

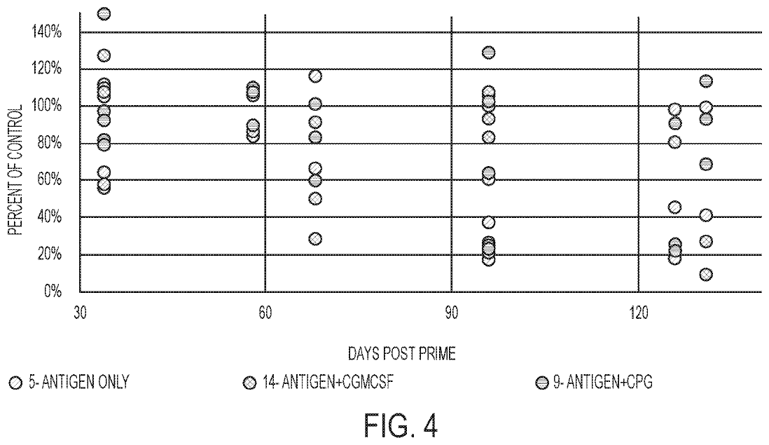

FIG. 4 shows testicular volumes, expressed as percent of age matched controls, compared with days after primary immunization. The number of mice with testicular volumes at or below 60% increased with time. Our histopathological observations indicate that mice with testicular volumes less than 30% of controls are functionally emasculated.

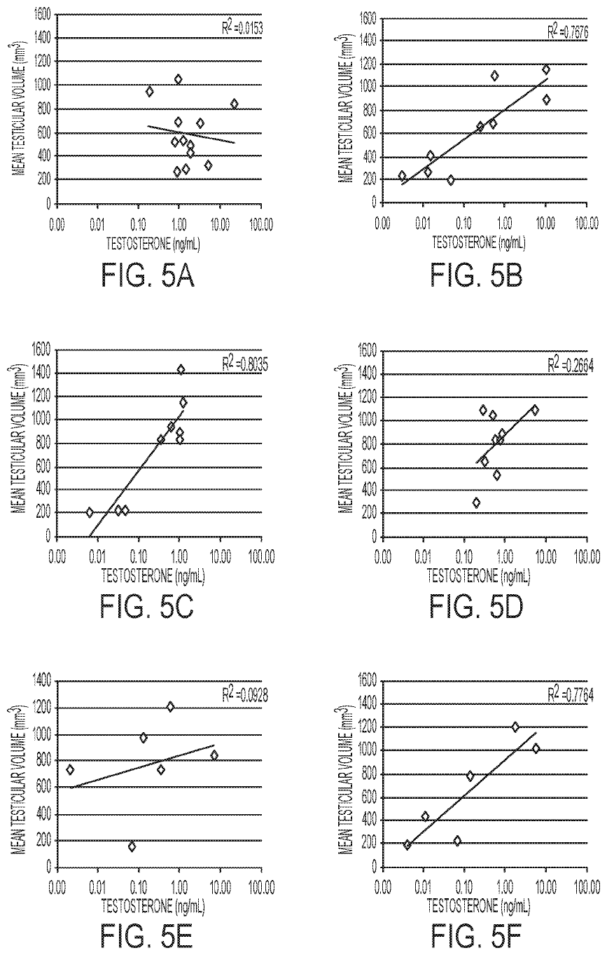

FIG. 5 shows a correlation between low testosterone and testicular volume for individual animals in 5 experiments at 90 days post-prime immunization, showing positive correlation between serum testosterone levels and testicular volume that is strongest for vaccines Ad-Leukotoxin-GnRH16Mouse (upper right panel) and Ad-Leukotoxin-GnRH16Dog (middle left panel) and Ad-LF-HBc-GnRH16 (lower right panel), but not for controls (upper left panel)

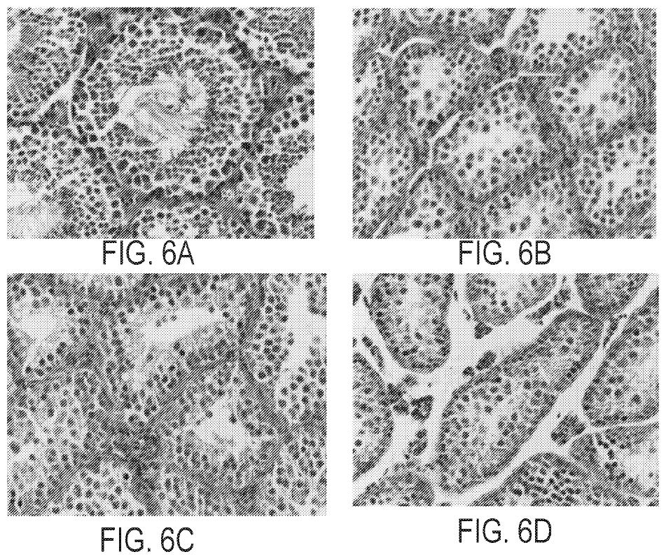

FIG. 6 shows histopathological changes in dysgenic testicles of immunized mice compared with normal testicular structure and spermiogenesis of CD1 mice.

FIG. 6A: Normal testicular histological structure showing well defined Leydig cells and spermiogenesis. FIG. 6B: Testicular dysgenesis showing loss of Leydig cells and aspermiogenesis in mice from antigen 9, 90 days post-primary immunization. FIG. 6C: Testicular dysgenesis showing loss of Leydig cells and aspermiogenesis (Ad-vectored GnRH vaccine #11, 60 days). FIG. 6D: Testicular dysgenesis showing loss of Leydig cells, intertubular inflammation and aspermiogenesis (Ad-vectored GnRH vaccine #12, 60 days). Magnification for all figures 25.2×.

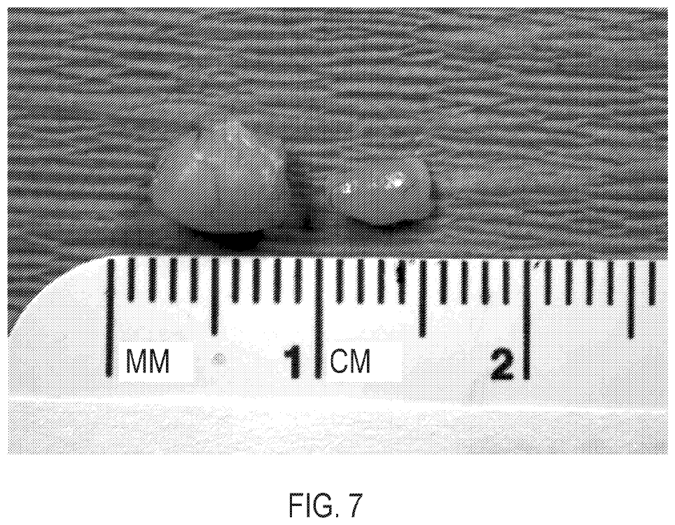

FIG. 7 shows gross necropsy appearance of a normal size testicle (left) compared with profound dysgenesis of an immunized mouse testicle.

Safety: All Ad-vectored GnRH vaccines purified by standard production protocols are safe. After immunization of mice with over 1000 doses of prime or booster injections there was no evidence of any adverse response locally or systemically. Body weights of immunized mice are normal and consistent with growth curve data for Charles River CD1 male mice used in these experiments. No lesions were observed in any organs at gross necropsy, except occasional bite wounds.

Antibody Response: Anti-GnRH antibody was evident from administration of 3 of 5 Ad-vectored GnRH vaccines tested at 30 days post-primary immunization. All geometric mean titers increased with time, reaching titers of over 10,000-fold over baseline with the Ad-Leukotoxin-GnRH16D vector. All the Ad-vectored GnRH vaccines produced titers in excess of 20-fold with the exception of Ad-PA83-GnRH16. There is a clear correspondence of high antibody titer and pronounced effects on the other contraceptive indices for Ad-Leukotoxin-GnRH16, Ad-LF-GnRH16 and Ad-LF-HBc-GnRH16.

Testosterone and Testicular Volumes: During the period from 30 to 90 days post-prime, there is a clear correlation between serum testosterone levels and testicular volume. This time-dependent correlation was characteristic of the most active vaccines (Ad-Leukotoxin-GnRH16 and Ad-LF-HBc-GnRH16) but, was not apparent in controls, or the Ad-PA83-GnRH16 or Ad-LF-GnRH16 groups.

Testicular Pathology: Gross testicular pathology was vaccination and post-vaccination time dependent. Diminution of testicular size was present by 60 days and increasingly frequent up to the last observation period of 120 days post-primary immunization. Histopathological changes are also time dependent. Decreased spermiogenesis leading to aspermiogenesis was observed along with necrosis of Leydig cells and a resultant intertubular inflammation. Microscopic evidence of complete aspermiogenesis is evident in testes with volumes less than 30% of controls. Based on these observations, we conclude that anti-GnRH antibodies block gonadotrophic stimulation causing testicular changes indicative of infertility to functionally emasculation.

Conclusions: All tested Ad-vectored GnRH vaccines are antigenic and contraceptive.

The five vaccines are all immunogenic and exhibit contraceptive activity in the mouse model. Three Ad-vectored GnRH vaccines induce GnRH antibodies within 30 days after primary vaccination. All constructs induced testicular dysplasia and resulted in significant lowering of testosterone levels in male mice. Two Ad-vectored GnRH vaccines including, Ad-Leukotoxin-GnRH16 (#8 and #9) and Ad-LFn-HBcGnRH16 (#12) appear superior to the others in achieving these effects in the mouse model.

Ad-LFn-HBcGnRH16 demonstrates strong effects on testosterone suppression and testicular dysgenesis.

AdsPA83GnRH16 and AdsLFnGnRH16 are immunogenic and contraceptive, but not as effective as the Ad-Leukotoxin-GnRH16 or Ad-LFn-HBc-GnRH16 vaccines.

### Example 3: Using GnRH Recombinant Adenovirus Vectors to Control Animal Populations and Suppress Undesirable Breeding Behavior

Use of Ad-Vectored GnRH Vaccines to Suppress Undesirable Estrus Behavior in Mares

Mares in estrus (heat) can be difficult to train. The intensity of behavioral estrus varies between mares and is an important factor in reduced athletic performance. Many mares become difficult to manage, show aggression, or perform irregularly during their estrus period. Suppressing this behavior is a very common request made to veterinarians by horse owners. There are several options to suppress estrus behavior in the mare, but all are constrained by high cost, are time consuming, inconvenient, and/or ineffective. Immunization against GnRH can inhibit its function and arrest the entire reproductive system. Antibodies produced against GnRH bind to endogenous GnRH preventing the stimulus required for release of FSH and LH. Cyclicity returns once the antibody titer falls below a threshold and normal reproductive function is restored. This reversible characteristic of GnRH vaccines is highly desirable for temporary suppression of behavioral estrus in mares. While current protein based GnRH vaccines can suppress ovarian function and cyclicity in mares, the duration of effect is variable (three months to greater than two years). These vaccines induce a high incidence of side effects such as injection site reaction and transient fever, which limits field use in performance mares. In addition, there are no commercial GnRH vaccines available for horses in the United States. Vaccination against GnRH using our novel Ad-vectored GnRH vaccine will suppresses reproductive function and estrus behavior. We will test our Ad-vectored GnRH vaccine in the mares to induce an immune response to GnRH that will temporarily stop the mare cycling, and suppress estrus behavior. See Example 5.

Use of Ad-Vectored GnRH Vaccines to Suppress Undesirable Breeding Behavior in Stallions

The expression of aggressive sexual behavior in performance stallions is a frequent and serious problem for owners and trainers. This behavior distracts from training and impedes performance. A stallion's full athletic potential is not appreciated until after he has reached puberty. This demonstrates a need to discover an effective, reliable, affordable, and reversible method to modify unwanted aggressive sexual behavior in stallions during the performance phase of their career. A successful adenoviral vectored vaccine will be adapted for use in stallions by stimulating the production of antibodies that suppress GnRH, reversibly suppress sexual behavior and testicular function in stallions, and will have no or minimal side effects associated with vaccine administration.

Use of Ad-Vectored GnRH Vaccine for Population Control of Wild Horses

The U.S. Bureau of Land Management is directed by the United States Congress to manage the American Wild Horse and Burro herds within authorized budgets on Federal lands. The size of these herds has reached the limits of currently authorized resources and unless there is dramatic control of their growth, these herds will far exceed any likely increases in allocated resources. Removal of stock has been a heroic, but unsuccessful solution because it has not been able to significantly alter the size of the herd, or inhibit the rate of herd growth and addresses the result rather than the cause of the problem. The cause is uncontrolled reproduction and the most ideal method of stabilizing and reducing the growth of these herds is non-surgical, field applicable contraception. Ad-vectored GnRH vaccines have a high potential for practical field application in controlling reproduction of wild horses and provide substantial advantages over other contraceptive vaccines, particularly anti-GnRH protein vaccines. Most of the past experience with anti-GnRH vaccines in horses has been based on protein formulations, which do not lend themselves to uniform, affordable mass production and a single dose as required for application to large numbers of wild horses. We have already achieved the basic design and proven the contraceptive effectiveness of Ad-vectored GnRH vaccines. Therefore, we will adapt these vaccines for immunization of horses (See Example 5) and formulation which incorporates all of the essential elements needed to test the immune response of horses to the Ad-vectored GnRH vaccine formulations and adapt these novel contraceptive vaccines for wild horse and burro population control.

Use of Ad-Vectored GnRH Vaccines for Population Control of Wild and Feral Animals

The frequency and danger of wild animal and human conflicts are rising at an alarming rate. The all too familiar statistics of damage done by deer illustrates the magnitude of this problem. Each year 1.5 million car accidents result from collisions with deer, costing 150 human lives, 10,000 human injuries and $1 billion in vehicle damage. The chance that an individual will contribute to this grim statistic is 1 in 150, odds for injury, death, of property loss are much too high. There are many other examples of damage done to crops by wild pigs, loss of pets and livestock by Coyotes, bite wounds caused by unwanted dogs and cats and diseases transmitted to domestic livestock and people, such as Lyme disease. With most of emerging infectious disease originating in wildlife, it is critical we better understand the importance of interactions between humans, livestock, wildlife, and our environment Killing these harmful animals attacks the result not the cause of this problem and despite the danger, is distasteful to the public. The cause is uncontrolled breeding and the only solution is an effective contraceptive. We will use our Ad-vectored GnRH vaccines that are proven to be effective in laboratory animals and mares (Example 5) for this application. These vaccines incorporate the latest advances in molecular biology and are equally effective in any animal species and either gender. That feature make this method effective in controlling populations of deer, coyotes, dogs, cats, pigs, etc without modification for each species.

Use of Ad-Vectored GnRH Vaccines for Population Control of Unwanted Dogs and Cats

In the early 1900s dog rabies was prevalent in the United States and remains a serious public health problem in much of the underdeveloped world. In response to this human health emergency, at the turn of the last century, U.S. municipal governments resorted to capture and kill for control of stray dogs. Invention of rabies vaccines solved the rabies problem, but not the unwanted dog and cat problem. Capture and kill remains the principal unwanted dog and cat control method, but policy is cruel, costly and ineffective because it attacks the result, but fails to addresses the cause; uncontrolled breeding. In the United States, low cost surgical neutering has shown limited impact, but surgery is not equal to the need for mass field application that is affordable and practical for use worldwide. Contraceptive vaccines that are effective, safe, marketable and affordable will succeed in solving the unwanted companion animal population problem by inducing long term infertility and cessation of objectionable breeding behavior of male and female dogs and cats. A correctly formulated anti-GnRH vaccine can induce antibodies which block the pituitary signaling of this hypothalamic hormone to release gonadotrophins, resulting in arrested sexual development of prepuberal dogs and cats and inducing infertility and cessation of breeding behavior when immunized after sexual maturity. In addition, activation of an appropriate cellular immune response may induce apoptosis of gonadal progenitor cells resulting in life-long cessation of sexual development, fertility and breeding behavior. Our studies demonstrate that properly formulated vaccines targeting gonadotropin-releasing hormone (GnRH), the apex hormone controlling reproduction, can disrupt fertility and suppress objectionable breeding behavior in dogs and cats for long periods, with no adverse effects on non-reproductive organs. Adenoviral vectors which express these successful anti-GnRH epitopes are key to rapid, robust and long term induction of a contraceptive immune response. We will optimize Ad-vectored GnRH vaccine which are effective, safe, inexpensive to manufacture and which meet the requirements for registration by the US Food and Drug Administration (FDA), which must be accomplished for wide spread use of the vaccine.

We will use Ad-vectored GnRH vaccines to induce long term sterility and block breeding behavior in both genders of domestic dogs and cats, after administration of a single dose, delivered by routine clinical methods adaptable to field use. The final Ad-vectored GnRH vaccine will satisfy regulatory requirements for defined constituents, quality control, efficacy and safety and performance in accordance with label claims.

### Example 4: Use of Adenoviral Vectored Gonadotropin Releasing Hormone Vaccine for Estrus Suppression of Mares

The goal of this study was to evaluate the effectiveness of an adenoviral vectored gonadotropin releasing hormone vaccine (Ad-GnRH) to suppress estrous cyclicity and unwanted estrous behaviour in mares. This study also evaluated a heterologous prime-boost immunization strategy to enhance an effective immune response. It was hypothesized a heterologous prime-boost vaccination strategy using an Ad-GnRH vector construct for the prime vaccine administration and a protein based GnRH vaccine boost would induce an antibody concentration above an effective estrous suppression threshold, compared with homologous Ad-GnRH prime and boost. Vaccine effects were measured by antibody production, ovarian activity, serum progesterone and estrous behaviour.

Ten normally estrous cycling mares were assigned to treatment (n=five) and control (n=five) groups. The experimental Ad-GnRH vaccine consisted of an adenoviral (Ad5, E1/E3 deleted) vector, engineered to express a GnRH antigen linked to a highly immunogenic, non-toxic “carrier” antigen (Bacillus anthracis lethal factor plus the core antigen of human hepatitis B). The Adenoviral vector construct was prepared essentially as disclosed in Example 1.

The mares in the treatment group were immunized intramuscularly twice, four weeks apart with the experimental Ad-GnRH vaccine. Eleven months following initial vaccination, all treatment mares received a heterologous boost using a low dose of a GnRH-protein vaccine (Equity® Oestrus Control Vaccine, Zoetis, Australia). Two additional mares were given the GnRH-protein conjugate to confirm that the low dose booster vaccine was not estrus suppressing itself.

Transrectal palpation and ultrasound of the reproductive tract was performed once or twice weekly for 16 months after initial vaccination. Mares were teased to a stallion for evaluation of estrous behaviour once to twice weekly. Venous blood was collected weekly to determine anti-GnRH antibody and serum progesterone concentrations. A full clinical examination was performed on mares twice daily for at least three days following vaccination to detect any adverse vaccine associated reactions. Serum progesterone concentration was measured by an electrochemiluminescence immunoassay. GnRH antibody was determined using a radio ligand binding assay. Results are presented as a proportion of an internal standard control. See FIG. 8.

Following the initial vaccination, all five control mares (100%) displayed normal cyclicity with an interestrus interval (IEI) mean±SEM of 23.29±2.28 days. Four of the treatment mares (80%) displayed normal cyclicity (IEI 22.75±2.41 days). One treatment mare showed two consecutive prolonged diestrus phases lasting 70 and 84 days. Seven weeks after heterologous boost, all treatment mares became acyclic, with minimal ovarian activity (largest follicle<25 mm, no corpus luteum), and serum progesterone concentrations maintained below 0.2 ng/ml. Estrous behaviour was erratic and inconsistent with displays of estrus, diestrus, and anestrus during each observation period so that a true inter-estrus interval could not be determined. All treated mares were still anestrous, with minimal ovarian activity and erratic estrous behaviour at the end of the 16-month study period. The low dose Equity® control mares continued to display normal cyclical estrous behaviour (IEI 21.24±3.2) days. Following heterologous boost, mean antibody response peaked at 4 weeks and remained elevated for the remainder of the study period.

The Ad-GnRH vaccinated mares increased GnRH antibody concentration significantly greater than that of control mares (P>0.05). Heterologous prime and boost vaccination increased antibody concentration above the threshold needed to suppress estrous cyclicity, induce anestrus and block most, but not all breeding behaviour. The study demonstrate the Ad-GnRH vaccine shows promise for controlled, reversible suppression of estrous behaviour, including when coupled with a heterologous anti-GnRH antigen.

### Example 5: Use of Adenoviral Vectored Gonadotropin Releasing Hormone Vaccine for Inducing Infertility in Mares

The objective of this study was to evaluate an adenoviral vectored gonadotropin releasing hormone (GnRH) vaccine as a method to suppress estrus behavior and cyclicity in mares. An additional objective was to determine the effects of a heterologous vaccination strategy against gonadotropin releasing hormone on estrus behaviour and cyclicity in mares using an adenoviral vectored gonadotropin releasing hormone vaccine to prime, and a protein based gonadotropin releasing hormone vaccine to boost. Twelve normal cyclic mares were included in the study which was divided into two phases. The first phase (weeks 0-46) included one ovulatory season and phase two (weeks 47-70) included the subsequent ovulatory season.

During phase one, treatment mares (n=5) were vaccinated twice, 4 weeks apart, with an adenoviral vectored gonadotropin releasing hormone vaccine. The vaccine was a replication-defective E1/E3 deleted adenovirus (Ad5) vector expressing antigens consisting of 16 multimers of GnRH, bacterial leukotoxin, T-helper epitopes, and other various hinge and linker amino acids (Ad-GnRH). Each 1 milliliter dose of the vaccine contained 4.64E10 infectious units. Five additional mares served as controls for estrus behavior, cyclicity, and seasonality. During phase two, mares that had been vaccinated during phase one (previous ovulatory season) were administered a single vaccination using a quarter of the labeled dose (sub-effective) of a protein based gonadotropin releasing hormone vaccine (100 μg protein conjugate per quarter of the labelled dose: Equity® Oestrous Control Vaccine, Zoetis, Australia). Two naïve mares (protein vaccine control mares) received an equivalent dose of the protein based gonadotropin releasing hormone vaccine to determine if the 100 μg protein conjugate dose was sub-effective for suppression of cyclicity and estrus. Anti-GnRH antibodies, estrus behavior, reproductive tract sonography, and serum progesterone concentrations were monitored over two consecutive breeding seasons.

Following homologous prime and boost using Ad-GnRH, all treatment mares developed anti-GnRH antibodies and the antibody response remained significantly different from that of time zero for 32 weeks during phase one. There was no effect on mean interestrus interval, reproductive cyclicity, or estrus behavior. Following heterologous boost during phase two, all treatment mares experienced an anti-GnRH antibody response that was maintained for at least 17 weeks (remainder of study period). All treatment mares became anestrus based on serum progesterone concentrations and transrectal sonographic findings. Estrus behavior became erratic and unpredictable, and therefore, interestrus interval could not be calculated. Protein vaccine control mares developed anti-GnRH antibodies after vaccination and the response was maintained for 15 weeks. There was no effect on interestrus interval, reproductive cyclicity, or estrus behavior. The day following Ad-GnRH boost, three of five treatment mares developed a non-painful 3-5 cm raised nodule at the injection site. Following heterologous boost, two of four treatment mares developed a small (<2 cm) raised, non-painful nodule. All injection sites reactions resolved within three days without treatment.

Homologous prime-boost vaccination utilizes the same vaccine formulation in both the prime and boost components of the regimen. Alternatively, heterologous prime-boost approaches use different vaccine formulations for the prime and boost injections. Heterologous prime-boost can therefore utilize different antigen delivery systems for induction of both humoral and cellular immunity. Recently, studies have shown that heterologous prime-boost regimens with a chimpanzee adenovirus type 7 viral vector expressing Human Immunodeficiency Virus (HIV) F4 fusion protein induced a polyfunctional HIV-1 specific CD4⁺ T-cell response in macaques (Lorin C. et al. Heterologous prime-boost regimens with a recombinant chimpanzee adenoviral vector and adjuvanted F4 protein elicit polyfunctional HIV-1-specific T-Cell responses in macaques. PLoS One 2015; 10:e0122835). Additionally, vector-prime protein-boost immunization induced broad hepatitis C virus-specific CD8+ and CD4+ T cell responses and functional Th1 type IgG responses in mice and guinea pigs (Chmielewska A. et al. Combined adenovirus vector and hepatitis C virus envelope protein prime-boost regimen elicits T cell and neutralizing antibody immune responses. J Virol 2014; 88:5502-5510). In that study, heterologous prime-boost induced an immune response that surpassed homologous vaccination alone. The study utilized a human adenovirus vector 6 expressing E1E2 glycoprotein as the priming vaccine, followed by recombinant protein vaccine (HVC genotype 1a E1E2p7) and MF59 adjuvant. Due to the small size and poor immunogenicity of GnRH, heterologous prime-boost vaccination utilizing a viral vector prime coupled with a protein based boost, offers the advantage of two separate antigen delivery systems to elicit an immune response.

This present study demonstrated that mares are capable of developing an anti-GnRH antibody response to homologous immunization using a replication-defective E1/E3 deleted replication-defective adenovirus vector encoding GnRH peptide, bacterial leukotoxin, and T-helper epitopes. Homologues prime-boost vaccination of mares with Ad-GnRH at the dose and frequency used in this study, however, did not result in suppression of reproductive cyclicity and estrus behavior.

The present study demonstrates that heterologous prime-boost vaccination of mares using an Ad-GnRH prime and protein based GnRH vaccine boost results in an antibody response that suppresses reproductive cyclicity, and interferes with estrus behavior. Vaccine-induced suppression of reproductive cyclicity and estrus was maintained for at least 17 weeks. See FIG. 10.

Twelve healthy, non-pregnant mares between 14-23 years of age with normal estrous cycles were included in the study. The mares were part of the Auburn University Equine Reproduction Center teaching herd. All mares were of average size (400-600 kg) and of various light horse breeds. Mares were housed by groups in large pens and were fed free choice coastal bermuda hay and supplemented with grain. Mares were examined via transrectal palpation and ultrasound to establish normal reproductive organ anatomy and ovarian activity (confirmed by the presence of a CL and an average interovulatory interval of 21 days). Additionally, all mares underwent a breeding soundness examination to establish normal reproductive health before inclusion in the study. Diagnostic procedures such as uterine culture and cytology where performed to ensure mares were free of endometritis and an endometrial biopsy was performed to ensure normal histoarchitecture. Finally, a complete blood count and serum biochemical analysis was performed for each mare to establish normal physiological health. Normal estrus behavior was confirmed via exposure to a stallion and characterized using the following scale:

One: Mare completely rejects the stallion, presenting one or more of the following refusal manifestations: squealing, pawing, kicking, switching tail, holding ears back.

Two: Mare is indifferent to the presence of the stallion; she does not move away, but does not lift the tail or display rhythmic eversion of the labia to expose the clitoris (clitoral eversion).

Three: Mare is interested in the stallion and approaches him, raising the tail, and everting the clitoris.

Four: Mare presents similar behavioral signs as score three: clitoral eversion, elevation of the tail, plus urination, change in posture to one that facilitates copulation (arched tail, flexed stifles and hock, abducted rear limbs and tipped pelvis with associated lowering of the perineal area).

Only healthy mares demonstrating regular estrous cycles, with normal uterine health, and normal estrus behavior in response to a teaser stallion were included in the study.

The Adenoviral vector construct was prepared essentially as disclosed in Example 1. An adenovirus 5 (Ad5) vector was constructed to encode a gene sequence that included human tissue plasminogen activator (tPA) leader sequence followed with 8 copies of GnRH (EHWSYGLRPG (SEQ ID NO: 17)) linked to LKT (leukotoxin A1 gene of Pasteurella haemolytica) followed by another 8 copies of GnRH. The amino acid trimers GSS or SGS were used as spacers between each of the GnRH monomers. The peptide TCPPCPAP was used as hinge sequence between each of the GnRH 8mers and the LKT sequence. Finally, the peptide MATVIDLS (SEQ ID NO: 18) was added between the hinge peptide and the N-terminus of the LKT. The infusion cassette was codon-optimized for dog cell expression, synthesized by GenScript (Piscataway, N.J.), and cloned into HindIII and XbaI sites of the pAdHigh vector (Altimmune) to generate pAdLKTGnRH16dog which was used for generation of AdLKTGnRH16dog vaccine virus as described before (Tang D, et al. Adenovirus as a carrier for the development of influenza virus-free avian influenza vaccines. Expert Rev Vaccines 2009; 8:469-481). AdLKTGnRH16dog vaccine virus was propagated on HEK293 cells and purified by ultracentrifugation over a cesium chloride gradient. The purified AdLKTGnRH16dog vector was sterilized by a 0.22-μm filtration then stored at −80° C. in a formulation buffer (Evans R, et al. Development of stable liquid formulations for adenovirus-based vaccines. J Pharm Sci. 2004; 93:2458-2475). AdLKTGnRH16dog viral titer was determined by Adeno-XTM rapid titer kit (BD Biosciences, Palo Alto, Calif.) on HEK293 cells. The correct structure of the AdLKTGnRH16dog antigen within the vector was verified by DNA sequencing (Genewiz, Germantown Md.). Each one ml dose of the vaccine contained 4.64E10 infectious units (ifu) of the vector. This vaccine will now be identified subsequently as Ad-GnRH.

Heterologous Boost Protein Based Vaccine Construct (Equity®)

The protein based GnRH vaccine was comprised of GnRH peptide conjugated to a diphtheria toxoid and admixed with an adjuvant immune-stimulating complex formed from Saponin Quil A, cholesterol, and dipalmitoylphosphatidycholine. Each 1.0 ml dose of this vaccine contained 200 μg peptide conjugate, 300 μg immunostimulating complexes, and 0.01% thimerosal, as a preservative, and isotonic buffered solution to volume (Equity® Oestrus Control Vaccine, Zoetis, Australia).

Experimental Treatment Phases

Phase 1 (week 0-46)

Treatment group: Five healthy, normally cyclic mares were vaccinated against GnRH by injection of one ml (4.64E10 ifu) of Ad-GnRH into the left cervical musculature twice, four weeks apart.

Control group: Five healthy, normally cyclic mares did not receive any treatments, and served as controls for reproductive cyclicity, estrus behavior, and seasonal changes in cyclicity.

Phase 2 (week 47-70)

Treatment group: Mares that were immunologically primed with the Ad-GnRH vaccine during phase one received a single vaccination with 0.5 ml of a protein based GnRH vaccine (100 μg peptide conjugate: one quarter of the recommended dose: Equity®) into the left cervical musculature 49 weeks after initial Ad-GnRH vaccination. One treatment mare that had been primed with Ad-GnRH mare was removed from phase two of the study and was euthanized for reasons unrelated to the study.

Protein vaccine control group: Two healthy, normally cyclic mares that were not included in phase one of the study (naïve mares) were vaccinated once with 0.5 ml of the protein based vaccine (100 μg peptide conjugate: Equity®) into the left cervical musculature. These control mares served to determine if vaccination using a single injection at an equivalent dose of that administered to treatment mares (100 μg peptide conjugate: Equity®) had an effect on reproductive cyclicity and estrus behaviour.

Anti-GnRH antibodies, estrus behavior, reproductive tract sonography, and serum progesterone concentrations were monitored over two consecutive breeding seasons. A venous blood sample was drawn from each mare immediately prior to initial injection with Ad-GnRH, and repeated weekly for the remainder of the study. Blood samples were collected by jugular venipuncture. Serum was centrifuged upon clotting at a rate 3000×g for 10 minutes. Sera was immediately separated, aliquoted, and frozen (−80° C.) until analysis. Data collection for this project ended 17 months after initial injection of treatment mares with Ad-GnRH.

Anti-GnRH Antibody Assay

Anti-GnRH antibody was detected from blood drawn every other week via binding of ¹²⁵I-labeled GnRH (L8008, Sigma, St Louis, Mo., USA) in serum using a radioimmuno-precipitation technique previously validated in mice and cats. Samples were assayed in duplicate and were performed as follows; 100 μl of ¹¹⁵I-labeled GnRH was added to 100 μl of test serum diluted 1:100, and 200 μl PBS/BSA (0.01 M phosphate buffer, 0.0027 M potassium chloride, 0.137 M sodium chloride, 1% (w/v) bovine serum albumin; pH 7.4 (Sigma-Aldrich). After overnight incubation at 4° C., 100 μl of bovine IgG PBS/BSA solution (total 250 μg bovine IgG) was added to each sample (19640, Sigma-Aldrich, St. Louis, Mo., USA). Bound ¹²⁵I-labeled GnRH was precipitated from unbound hormone by adding 500 μl of a 24% solution of polyethylene glycol (PEG; Carbowax™ PEG 8000, P156-500, Thermo Fisher Scientific, Waltham, Mass., USA). Tubes were then vortexed and incubated at 4° C. for 10 minutes. Tubes were centrifuged at 1400×g for 15 minutes and the supernatant was aspirated. Radioactivity in the pellet was measured in a gamma counter. Nonspecific binding (NSB) of ¹²⁵I-labeled GnRH was determined from the mean of duplicate tubes in which the diluted serum was replaced by PBS/BSA buffer. Mean NSB was subtracted from individual sample measurements. The serum anti-GnRH antibody that was bound to ¹¹⁵I-labeled GnRH was expressed as a proportion of an internal standard control.

Progesterone

Serum progesterone concentration was assayed weekly and was analyzed using a chemiluminescence immunoassay, Immulite® Progesterone (Diagnostic Products Corporation, Los Angeles, Calif.).

Transrectal Palpation and Ultrasound of the Reproductive Tract

The reproductive tract of all mares was examined via transrectal palpation and ultrasound with a 5 MHz linear array transducer (Sonosite, Sonosite Inc., Bothell, Wash.). During phase one (week 0-46), sonographic examination was performed twice weekly during the summer and early fall (week 0-25). During the subsequent winter (from week 35), sonographic examinations were performed once to twice weekly.

During phase 2 (week 47-70), sonographic examinations were performed twice weekly for the remainder of the study. At each examination, the presence or absence of a CL was noted. Uterine edema was classified as follows: No uterine edema (score 0), slight uterine edema (score 1), moderate uterine edema (score 2), and heavy uterine edema (score 3). The diameter of the largest follicle on each ovary was measured and these values were used to calculate the anestrus index.

Estrus Behavior and Interestrus Interval

Estrus behavior was assessed on the same schedule as sonographic examinations. A teaser stallion was led to the paddock and the mares allowed to approach and make contact through a fence. If a mare did not approach the fence, she was haltered and led to the fence line to ensure contact with the stallion. Estrus behavior was scored using the same scale described in the mare inclusion criteria. Mares with an assigned teasing score of one or two were considered in either anestrus or diestrus, and those that were assigned a teasing score of three or four were considered to be in estrus. Interestrus interval (IEI) was calculated as the time period from when a mare first displays an estrus score of 3 or more followed by scores consistent with diestrus/anestrus for two or more days, to when the mare next first displayed an estrus score of 3 or more when presented with a stallion.

Anestrus Index

Anestrus index was assigned to identify behavior consistent with anestrus. Anestrus index was based on scores assigned for parameters of sonographic findings of the reproductive tract, serum progesterone concentration, and estrus behavior. A score of 3 was assigned for parameters consistent with anestrus, while a score of 0 was assigned for a parameter that was not consistent with anestrus, and included parameters that represent both diestrus and estrus. Measured parameters included tease score (>2 not anestrus), largest follicle diameter (≥25 mm not anestrus), uterine edema score (≥1 not anestrus), the presence of a corpus luteum (CL), and serum progesterone concentration (≥2 ng/ml not anestrus). The table below shows the allocation of score for anestrus index parameters.

| | |
|---|---|
| Score 0 | Score 3 |

| | | |
|---|---|---|
| Tease Score | 2 or less | >2 |

| | | | | |
|---|---|---|---|---|
| Largest follicle diameter | ≥25 | mm | <25 | mm |

| | | |
|---|---|---|
| Corpus Luteum | Present | Absent |
| Uterine edema score | <1 | ≥1 |

| | | | | |
|---|---|---|---|---|
| Serum progesterone | ≥2 | ng/ml | <2 | ng/ml |

For example, a mare with a tease score of 2 or less, largest follicle size less than 25 mm, no CL or uterine edema noted during sonographic examination, and base line serum progesterone concentration would receive a score of 3 for each parameter, and therefore a cumulative score of 3+3+3+3=12. Alternatively, a mare with a tease score of 3 or more, largest follicle greater than 25 mm, no CL noted on sonographic examination, and a uterine edema score of 3 would receive a score of zero for each parameter, and therefore a cumulative score of 0+0+0+0=0. A total score greater than 10 was considered consistent with anestrus (acyclic), while a score less than 10 was considered consistent estrus or diestrus (cyclic).

Anti-GnRH Antibody

Phase One (Week 0-46): Ad-GnRH Prime Day 0, Ad-GnRH Boost Week 4

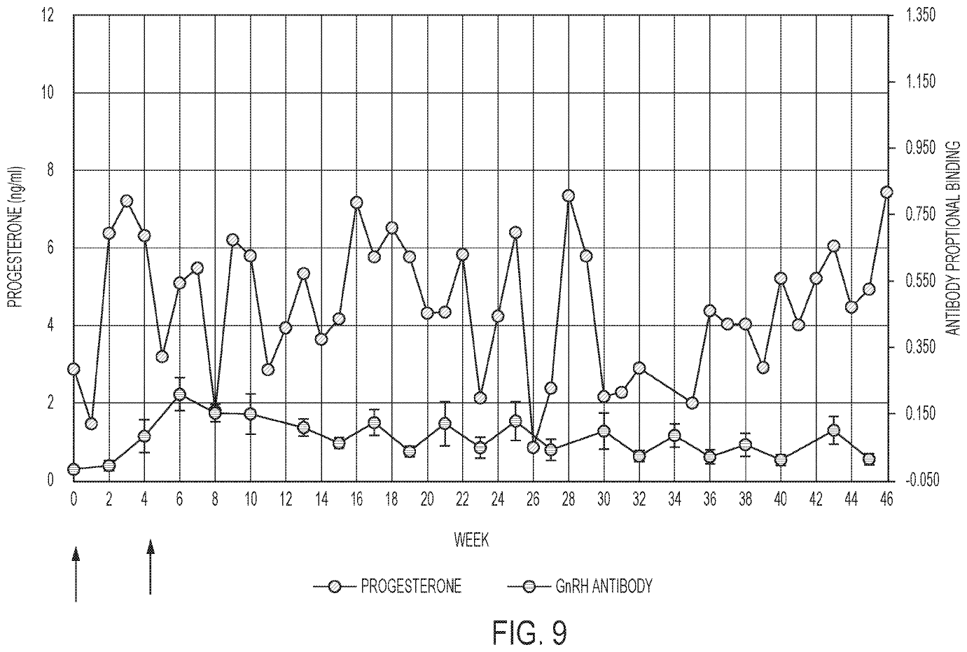

All mares were seronegative for GnRH antibodies prior to first vaccination. All mares were considered seronegative when antibody radioligand binding as a proportion of the internal standard (PBIS) was less than 0.0066 (based on a day 0 PBIS value treatment group mean of −0.0174±standard error of the mean). All Ad-GnRH vaccinated mares responded with the production of anti-GnRH antibodies. Antibodies were detectable in all vaccinated mares at the time of the second boost (week 4). At the first occurrence that anti-GnRH antibodies could be detected, mean antibody production for treatment mares, as measured by PBIS, was 0.0825±0.0498. This was significantly different from that of time zero (P=0.01). Peak mean anti-GnRH antibody response in treatment mares occurred 6 weeks after homologous Ad-GnRH prime (range 6-25 weeks). The PBIS value at this time was 0.2087±0.0263, which is 12.5 times that of the upper end of the confidence limit from what was considered negative at time zero. Antibody response gradually waned until it decreased to a value that was considered negative for anti-GnRH antibody at week 36 weeks after Ad-GnRH prime. See FIG. 9.

Phase Two (Week 47-70): Heterologous Boost with Protein Antigen at Week 49

At the beginning of phase two, which was 47 weeks after Ad-GnRH prime, treatment mare mean anti-GnRH antibody PBIS value was 0.01058±0.0220. This was not significantly different from the value of 0.0174±0.0086 that was considered negative for anti-GnRH antibody at time zero (P=0.25). Three weeks following heterologous boost (week 49 after Ad-GnRH prime), a significant increase in antibody response of treatment mares was detected (P=0.01). At this time, the mean PBIS value for treatment mares was 0.5945±0.0582. This value is 90 times greater than the upper limit of the confidence interval for what was considered negative for Anti-GnRH antibodies. Maximum mean antibody response from treatment mares occurred five weeks after heterologous boost (54 weeks after Ad-GnRH prime), with a PBIS value of 0.8234±0.1798. This peak was 124.75 times higher than the upper limit for the confidence interval for what was considered negative at time zero. Mean anti-GnRH antibody response for treatment mares remained significantly higher than that of time zero for the remainder of the study period. See FIG. 10.

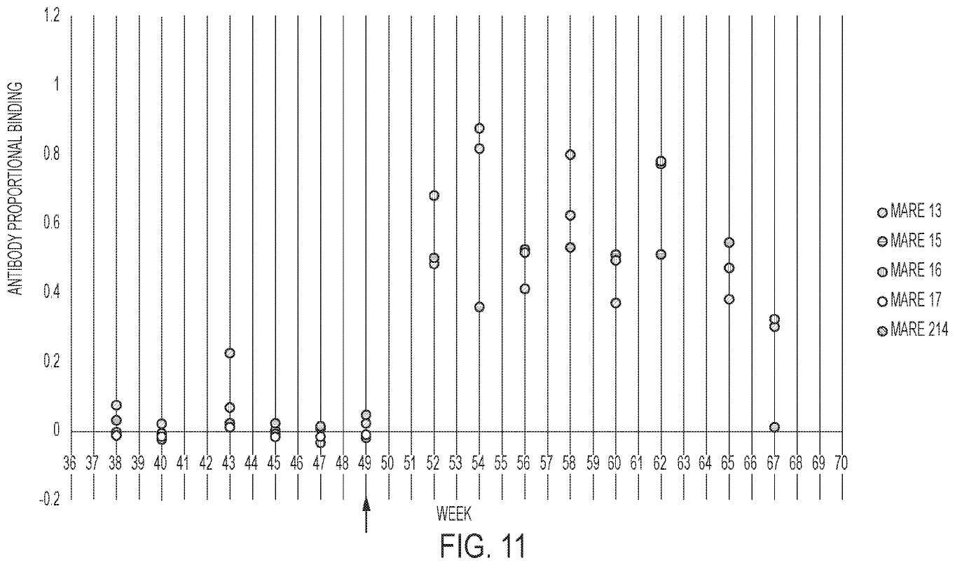

Anti-GnRH antibodies were detectable for protein vaccine control mares three weeks after vaccination (Week 52). The PBIS value representing the mean anti-GnRH antibody response for these two mares was 0.11048±0.0659, which is 16.749 times greater than what was considered negative for treatment mares at the beginning of phase one. Peak anti-GnRH response for these protein vaccine control mares occurred five (mare 210) and seven (mare 211) weeks post vaccination. These PBIS values were 0.15135 and 0.0191 respectively (FIGS. 4a, 4b). By 18 weeks post vaccination (week 67), anti-GnRH antibody response for protein vaccine control mares decreased to −0.01616 and was no longer different from what was considered negative for treatment mares. There was a wide frequency distribution of antibody responses for treatment mares, especially following heterologous boost with the protein based GnRH vaccine. See FIG. 11.

Progesterone

Phase One (Week 0-46): Ad-GnRH Prime Day 0, Ad-GnRH Boost Week 4

Serum progesterone concentration for all mares displayed cyclical changes throughout the study period that reflected a normal interovulatory interval. Furthermore, following exposure to a stallion, all mares showed diestrus behavior when serum progesterone concentration was greater than 2 ng/ml, and estrus behavior when serum progesterone concentration was less than 2 ng/ml.

Phase Two (Week 47-70): Heterologous boost with protein antigen at week 49

At the beginning of phase two, serum progesterone concentration for all mares displayed cyclical changes that reflected a normal interovulatory interval. Three weeks following the heterologous boost (52 weeks after Ad-GnRH priming), serum progesterone concentrations for all treatment mares were below 2 ng/ml. Serum progesterone concentration for this group of mares remained below 2 ng/ml for the remainder of the study period. Serum progesterone concentrations for protein vaccine control mares reflected normal cyclicity throughout the study period.

Interestrus Interval

Phase One (Week 0-46): Ad-GnRH Prime Day 0, Ad-GnRH Boost Week 4

Four of the five treatment mares displayed normal interestrus intervals (IEI), with a mean IEI of 23±2 days, which was not different from control mares (mean IEI 22±2 days) (P=0.462). One treatment mare (Mare 214) experienced two prolonged luteal phases of 70 days and 91 days respectively. This mare was determined as an outlier because these interestrus intervals were greater than 33 days, which is 1.5 times the interquartile range above the third quartile of the data. Data for this mare was not considered for statistical analysis of IEI.

Phase Two (Week 47-70): Heterologous Boost with Protein Antigen at Week 49

At the beginning of phase two, prior to heterologous boost with a protein antigen, four of 5 treatment mares displayed normal IEIs (25±4 days). The same mare that had experienced prolonged luteal activity during phase one (mare 214) also experienced prolonged luteal activity that extended into phase two. The mare's luteal phase lasted 85 days and occurred from week 38 through week 50. The duration of the luteal phase was greater than 68 days, which is 1.5 times the interquartile range above the third quartile of the data. Again, because this mare was determined to be an outlier, data for this mare was not considered for statistical analysis of IEI.

Four weeks following heterologous boost, treatment mares ceased displaying predictable estrus that would allow for calculation of IEI. At each observation period, teasing behavior became erratic. Mares would display behaviors consistent with estrus (score 3, 4) and anestrus/diestrus (1, 2) during the same observation period. IEI for treatment mares could not be calculated for the remainder of the study period (weeks 53-70). One mare that appeared to return to cyclicity (Mare 15) based on anestrus index score did not complete one full IEI before the completion of the study to allow for calculation of one IEI.

Protein vaccine control mares exhibited normal interestrus intervals (27±3 days) that were not different from interestrus intervals determined from control mares during phase one of the study.

Anestrus Index

Phase One (Week 0-46): Ad-GnRH Prime Day 0, Ad-GnRH Boost Week 4

Anestrus index for treatment mares was calculated from measures of mare cyclicity taken from weeks 0-25, and again from weeks 35-46. All vaccinated mares remained cyclic following homologous Ad-GnRH prime and boost vaccination (anestrus score<10). Four of five control mares exhibited normal reproductive cyclicity throughout the time period for which their cyclicity was monitored (weeks 11-25). One control mare (mare 11) cycled normally until week 16, after which the mare became anestrus (anestrus scores maintained above 10) for the remainder of the observation period for Ad-GnRH control mares. This occurred during late September to early October and may reflect normal seasonal transition.

Phase Two (Week 47-70): Heterologous Boost with Protein Antigen at Week 49

All treatment mares exhibited normal reproductive cyclicity at the beginning of phase two (anestrus score<10), prior to heterologous boost. Four weeks following heterologous boost (week 53), all treatment mares became acyclic (anestrus index score>10). All mares remained anestrus until week 68, after which one mare (Mare 15) returned to cyclicity based on the anestrus index score (anestrus index score<10) (FIG. 11). This mare developed a large dominant follicle>30 mm, uterine edema score>1, estrus behavior score>2, and progesterone was <0.2 ng/ml. The mare did not ovulate before the end of the study period. Protein vaccine control mares exhibited normal reproductive cyclicity (anestrus index score<10) for the duration that their cyclicity was monitored (weeks 49-70).

Homologous prime-boost immunization against GnRH using the experimental Ad-GnRH vaccine resulted in the production of anti-GnRH antibodies in all treated mares. Heterologous prime-boost vaccination resulted in greater production of anti-GnRH antibodies compared with that of initial homologous prime-boost vaccination. The present study used only five mares during phase one, and four treatment mares during phase two. In spite of the limited number of mares, it is clearly shown that immunization of mares against GnRH utilizing a vaccine strategy that incorporates Ad-GnRH prime and a heterologous protein antigen boost protocol results in the production of anti-GnRH antibodies and concurrent suspension of reproductive cyclicity and estrus behavior. This study demonstrates that mares are capable of developing an anti-GnRH antibody response to homologous immunization using a replication-defective E1/E3 deleted replication-defective adenovirus vector encoding GnRH peptide, bacterial leukotoxin, and T-helper epitopes. Homologues prime-boost vaccination of mares with Ad-GnRH at the dose and frequency used in this study does not result in suppression of reproductive cyclicity and estrus behavior. This study demonstrates that heterologous prime-boost vaccination of mares using an Ad-GnRH prime and protein based GnRH vaccine boost results in an antibody response that suppresses reproductive cyclicity, and interferes with estrus behavior. Vaccine induced effects are observed within four weeks of heterologous boost and may be maintained for at least 12 weeks.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An immunogenic composition comprising:
an adenovirus vector that contains and expresses a nucleic acid encoding a recombinant protein, comprising an immunogenic carrier antigen, and endogenous mammalian GnRH or homolog thereof.

2. The immunogenic composition of claim 1, wherein the adenovirus vector comprises linear repeats of GnRH.

3. The immunogenic composition of claim 2, wherein the adenovirus vector comprises about 6 to about 20 repeats of GnRH.

4. The immunogenic composition of claim 1, wherein the carrier antigen is flanked by linear repeats of GnRH.

5. The immunogenic composition of claim 4, wherein the carrier antigen is flanked by about 6 to 10 linear repeats of GnRH.

6. The immunogenic composition of claim 2, wherein the linear repeats of GnRH are separated by a linker encoding 3 to 6 amino acids.

7. The immunogenic composition of claim 1, wherein the adenovirus vector is selected from an E1, E3, and/or E4 deleted or disrupted adenovirus.

8. The immunogenic composition of claim 1, wherein the adenovirus vector is replication deficient.

9. The immunogenic composition of claim 1, wherein the recombinant protein comprises T cell epitopes.

10. The immunogenic composition of claim 1, wherein the carrier antigen comprises a bacterial or viral immunogenic antigen, or immunogenic fragment thereof.

11. The immunogenic composition of claim 1, wherein the carrier antigen comprises leukotoxin antigen, B. anthracis lethal factor, B. anthracis protective antigen, tetanus toxin, diphtheria toxin, Hepatitis B core antigen, or a combination thereof.

12. The immunogenic composition of claim 11, wherein B. anthracis protective antigen is PA83, PA63 or an immunogenic fragment thereof.

13-24. (canceled)

25. An immunogenic formulation comprising, the immunogenic composition of claim 1 and an adjuvant.

26. (canceled)

27. The immunogenic formulation of claim 25, wherein the formulation is a liquid, a solid, lyophilized, or a suspension.

28. (canceled)

29. A method for inducing an immune response against GnRH in a mammal comprising, administering the immunogenic composition of claim 1.

30. The method of claim 29, wherein the mammal is a companion animal, a domesticated animal, a feral animal, a food-or feed-producing animal, a livestock animal, a game animal, a racing animal, a performance animal, or a sport animal.

31. (canceled)

32. The method of claim 29, wherein administration is intradermal, subcutaneous, intramuscular, oral, topical. intravenous or intranasal.

33. (canceled)

34. The method of claim 29, wherein the immune response against GnRH induces infertility.

35. (canceled)

36. The method of claim 29, comprising a homologous prime-boost dosing regimen.

37. The method of claim 29, comprising a heterologous prime-boost dosing regimen.

38-64. (canceled)
